(19) United States
(12) Patent Application Publication
(10) Pub. No.: US 2020/0087326 A1
(43) Pub. Date: Mar. 19, 2020

(54) DEEP RED FLUORESCENT PROBE

(71) Applicant: The University of Tokyo, Tokyo (JP)

(72) Inventors: Kenjiro HANAOKA, Tokyo (JP); Yasuteru URANO, Tokyo (JP); Koji NUMASAWA, Tokyo (JP); Takayuki IKENO, Tokyo (JP); Yuki HOSHINO, Tokyo (JP)

(21) Appl. No.: 16/466,002

(22) PCT Filed: Dec. 1, 2017
(86) PCT No.: PCT/JP2017/043240

§ 371 Date: Nov. 19, 2019

Related U.S. Application Data
(60) Provisional application No. 62/428,662, filed on Dec. 1, 2016.

(51) Int. Cl. C07F 7/08; A61K 49/00; C09K 11/06; C09B 11/24

(52) U.S. Cl. CPC C07F 7/0816; A61K 49/0041; C09K 2211/1007; C09B 11/24; C09K 2211/1018; C09K 11/06

(57) ABSTRACT

A near-infrared fluorescent probe has fluorescence in the near-infrared region. Like CaSiR-1, the probe has rhodamines as the fluorescent mother nucleus and accumulates in the cytoplasm. The probe makes it possible to visualize concentration fluctuations in metal ions, such as calcium ions, within the body. The fluorescent probe includes a compound represented by the following general formula or a salt of the compound:

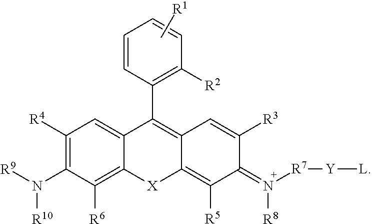

### TECHNICAL FIELD

The present invention relates to a novel fluorescent probe, more specifically to a novel deep red fluorescent probe.

### BACKGROUND ART

Calcium ions (Ca²⁺) play an important role in the body as a second messenger (Non-Patent Document 1). Under physiological conditions, the Ca²⁺ concentration of the cytoplasm is kept low; i.e., up to 100 nM, but Ca²⁺ flows into the cytoplasm from outside the cell or the endoplasmic reticulum (ER), mitochondria, etc., in response to stimulation and elicits various biological responses by interacting with Ca²⁺-binding proteins such as calmodulin. In particular, fluctuations in the Ca²⁺ concentration in the cytoplasm are involved in the regulatory mechanisms of a wide range of life phenomena such as contraction of muscles such as the myocardium and skeletal muscles, spontaneous firing associated with neurotransmission, and enzyme secretion in the pancreas, and tools for tracking calcium concentration fluctuations in the cytoplasm are very important in biological research.

Visualization using Ca²⁺ imaging probes has been the method for tracking time-dependent concentration fluctuations up to the present. FIG. 1 shows examples of widely used fluorescent probes having a xanthene dye as the mother nucleus (Non-Patent. Documents 2 and 3).

Ca²⁺ fluorescent probes comprise a fluorophore site and a chelator site called BAPTA (1,2-bis(o-aminophenoxy)ethane-N,N,N′,N′-tetraacetic acid) which undergoes coordination-bonding with Ca²⁺. Except for Rhod-2, fluorescein derivatives are used in the fluorescent mother nucleus. These probes characteristically accumulate in the cytoplasm and are characteristically suited to sensitive detection of the Ca²⁺ involved in physiological functions within the cell.

Rhod-2, which has rhodamine as the mother nucleus, has a longer wavelength than probes having fluorescein as the mother nucleus. This probe, however, is used to measure the mitochondrial Ca²⁺ because due to exhibiting mitochondrial localization, unlike other rhodamines.

In recent years, the present inventors developed the Ca²⁺ probes CaTM-2 (Non-Patent Document 4) and CaSiR-1 (Non-Patent Document 5) which have as the mother nucleus a dye in which the O atom of the xanthene ring position 10 has been substituted by an Si atom. CaTM-2, which has a fluorescein analog as the fluorophore, has a red fluorescence and accumulates in the cytoplasm. CaSiR-1, which has Si-rhodamine as the fluorophore, has fluorescence in the near-infrared region, but exhibits lysosomal localization.

As described above, a probe having a fluorescein analog as the fluorescent mother nucleus must be used to visualize calcium concentration fluctuations in the cytoplasm, which trigger various physiological events, and probes having rhodamines as the mother nucleus are used to observe calcium concentration fluctuations in various organelles.

### PRIOR ART REFERENCES

### Non-Patent References

Non-Patent Document 1: Clapham D. E., Cell, 2007, 131, 1047-1058.

Non-Patent Document 2: Minta A., Kao J. P. Y., Tsein R. Y., J. Biol. Chem., 1989, 264, 8171.

Non-Patent Document 3: Johnson I., Spence M. T. Z., Ed. The Molecular Probes Handbook: A Guide to Fluorescent Probes and Labeling Technologies, 11^{th} Ed. Molecular Probes, Inc. 2010.

Non-Patent Document 4: Egawa T., Hirabayashi K., Koide Y., Kobayashi C., Takahashi N., Mineno T., Terai T., Ueno T., Komatsu T., Ikegaya Y., Matsuki N., Nagano T., Hanaoka K., Angew. Chem. Int. Ed., 2013, 52, 3874-3877.

Non-Patent Document 5: Egawa T., Hanaoka K., Koide F. Ujita S., Takahashi N., Ikegaya Y., Matsuki N., Terai T., Ueno T., Komatsu T., Nagano T., J. Am. Chem. Soc., 2011, 133, 14157-14159.

### SUMMARY OF THE INVENTION

### Problems to be Solved by the Invention

It is an object of the present invention to provide a novel near-infrared fluorescent probe that accumulates in the cytoplasm.

### Means Used to Solve the Above-Mentioned Problems

Rhodamines, which are characterized by magnitude of their wavelength, make it possible to develop probes having fluorescence in the near-infrared region that could not be attained by fluorescein analogs, and can provide new color windows in multicolor imaging.

The present inventors therefore conducted studies to develop a commercially viable fluorescent probe that has fluorescence in the near-infrared region, as with CaSiR-1, which has rhodamines as the fluorescent mother nucleus and that accumulates in the cytoplasm and makes it possible to visualize concentration fluctuations in, inter alia, metal ions such as calcium ions within the body.

Ca²⁺ probes having rhodamines as the fluorescent mother nucleus exhibit accumulation in the mitochondria and lysosomes due to the cationicity of the xanthene ring and cannot be made to accumulate in the cytoplasm where there are large fluctuations in the intracellular concentration of metal ions such as Ca²⁺ within the body. The present inventors therefore suppressed accumulation in specific intracellular organelles such as the mitochondria derived from cationicity by making the overall charge of the fluorescent dye molecule be 0 as a molecular design, considered the possibility of developing rhodamines of Si, etc. to remain more in the cytoplasm, and introduced anionic functional groups of carboxylic acids, etc., at benzene ring sites.

The present inventors also thought that a Ca²⁺ probe that exhibits cytoplasmic accumulation could be developed by bonding a structure in which a carboxylic acid of the BAPTA structure known as a Ca²⁺ chelator had been protected by an acetoxymethyl group (AM group) with rhodamine and synthesized various compounds in which rhodamine dyes were bonded with BAPTA structures. As a result, the inventors discovered that compounds bonded via a linker extended from a nitrogen atom of the xanthene ring exhibit a high S/N ratio and thereby perfected the present invention.

Specifically, the present invention provides:

[1] A compound represented by the following general formula (I) or a salt thereof:

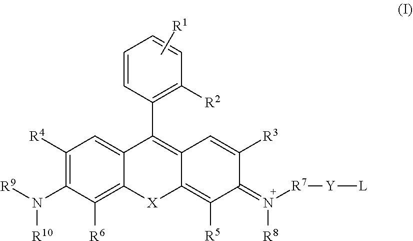

where:
R¹ is a hydrogen atom or one to four of the same or different monovalent substituents present on the benzene ring, and R¹ may be the same or different;
R² is an anionic functional group, a C1-10 alkyl group, or a C1-10 alkoxy group;
R³ and R⁴ are, each independently, a hydrogen atom, a C1-6 alkyl group, or a halogen atom;
R⁵ and R⁶ are, each independently, a hydrogen atom, a C1-6 alkyl group, or a halogen atom;
X is SiR¹¹R¹², GeR¹¹R¹², SnR¹¹R¹², CR¹¹R¹², SO₂, or POR¹³,
   R¹¹ and R¹² are, each independently, a C1-6 alkyl group or as aryl group,
   R¹³ is a C1-6 alkyl group or an optionally substituted phenyl group;
R⁷ is a C1-6 alkylene group;
R⁸ is a hydrogen atom or a C1-6 alkyl group,
R⁸ optionally forms, together with R⁵, a five- to seven-membered heterocyclyl or heteroaryl containing a nitrogen atom to which R⁸ is bonded, optionally containing one to three heteroatoms selected from the group consisting of an oxygen atom, nitrogen atom, and sulfur atom as ring members, and the heterocyclyl or heteroaryl may be substituted by a C1-6 alkyl, C2-6 alkenyl, or C2-6 alkynyl, C6-10 aralkyl group, or C6-10 alkyl-substituted alkenyl group;
R⁹ and R¹⁰ each independently represent a hydrogen atom or a C1-6 alkyl group,
R⁹ and R¹⁰ together may form a four- to seven-membered heterocyclyl containing a nitrogen atom to which R⁹ and R¹⁰ are bonded,
R⁹ or R¹⁰, or both R⁹ and R¹⁰, together with R⁴, R⁶, respectively, may form a five- to seven-membered heterocylyl or heteroaryl containing a nitrogen atom to which R⁹, R¹⁰ are bonded, may contain from one to three heteroatoms selected from the group consisting of an oxygen atom, nitrogen atom, and sulfur atom as ring members, and the heterocyclyl or heteroaryl may be substituted by a C1-6 alkyl, C2-6 alkenyl, or C2-6 alkynyl, C6-10 aralkyl group, or C6-10 alkyl-substituted alkenyl group;
Y, when present, is a spacer:
L is a substituent which acts as a capturing group for a substance to be measured.

[2] The compound or salt thereof according to [1], wherein the anionic functional group of R² is selected from a hydroxyl group, carboxy group, sulfo group, C1-10 hydroxyalkyl group, C1-10 alkyl group having a carboxy group, or C1-10 alkoxy group having a carboxy group.

[3] The compound or salt thereof according to [1] or [2], wherein the capturing group is a capturing group for capturing a proton, a metal ion, a low-oxygen environment, an active oxygen species, nitrogen monoxide, hydrogen peroxide, singlet oxygen, or a pH environment.

[4] The compound or salt thereof according to [3], wherein the metal ion is selected from a zinc ion, magnesium ion, sodium ion, potassium ion, or calcium ion.

[5] The compound or salt thereof according to any one of [1] [4], wherein the capturing group is a capturing group for capturing a calcium ion.

[6] The compound or salt thereof according to any one of [1]-[5], wherein Y is an amide, ester, or thiourea.

[7] The compound or salt thereof according to any one of [1]-[6] wherein L is a capturing group for capturing a calcium ion represented by general formula (1) below.

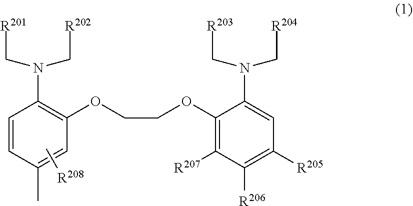

wherein, R²⁰¹, R²⁰², R²⁰³, and R²⁰⁴ are, each independently, a carboxy group, an alkyl group having a carboxy group, an ester group, an optionally substituted alkyl ester group, or a salt thereof;
R²⁰⁵, R²⁰⁶, and R²⁰⁷ are, each independently, a hydrogen atom, a halogen atom, a C1-6 alkyl group, a methoxy group, or a nitro group;
R²⁰⁸ is a hydrogen atom or represents from one to three of the same or different monovalent substituents present on the benzene ring.

[8] The compound or salt thereof according to any one of [1]-[7], wherein L is a capturing group for capturing a calcium ion represented by formula below:

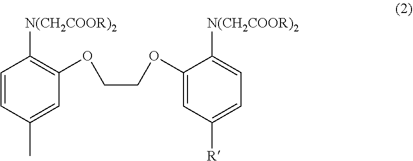

wherein, R is hydrogen or —CH₂OCOCH₃, each R may be the same or different:
R′ is a methyl group, a methoxy group, or a fluorine atom).

[9] The compound or salt thereof according to any one of [1]-[8], wherein R² is a carboxy group.

[10] The compound or salt thereof according to any one of [1]-[9], wherein R⁷ is selected from a methylene group or an ethylene group and R⁸ is selected from a methyl group or an ethyl group.

[11] The compound or salt thereof according to any one of [1]-[10], wherein R⁹ and R¹⁰ are, each independently, selected from a methyl group or an ethyl group.

[12] The compound or salt thereof according to any one of [¹]-[¹¹], wherein R⁷ is a methylene group, and R⁸, R⁹, and R¹⁰ are all methyl groups.

[13] The compound or salt thereof according to any one of [1]-[12], wherein R¹ are all hydrogen atoms.

[14] A compound represented by formula (3) below, or a salt thereof.

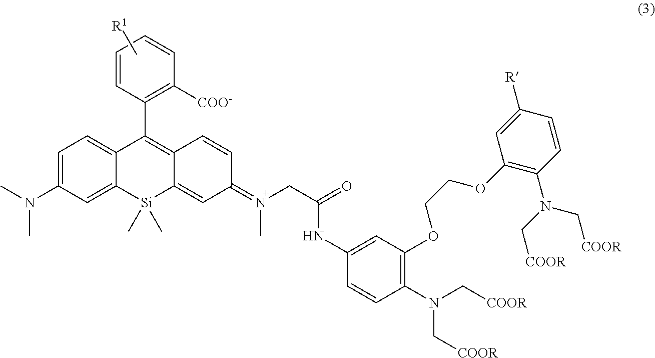

wherein, R is hydrogen or —CH₂OCOCH₃, each R may be the same or different:

R′ is a methyl group, a methoxy group, or a fluorine atom, R¹ is as defined in general formula (I).

[15] A fluorescent probe containing a compound or salt thereof according to any one of [1]-[14].

[16] A method for measuring a substance to be measured, wherein the method comprises the steps of:

(a) bringing the compound or salt thereof according to any one of [1]-[15] into contact with a substance to be measured and

(b) measuring the fluorescence intensity of the compound after capture of the substance to be measured generated in step (a).

[17] The method according to [16], wherein the substance to be measured is a calcium ion.

### Advantages of the Invention

The present invention can provide a near-infrared fluorescent probe that accumulates in the cytoplasm.

The present invention can also provide a calcium fluorescent probe that exhibits high cytoplasmic accumulation and a high S/N ratio even in live cell imaging by bonding a structure in which a carboxylic acid of the Ca²⁺ chelator BAPTA structure has been protected by an acetoxymethyl group (AM group) via a linker extended from a nitrogen atom of the xanthene ring of a rhodamine dye.

### BRIEF DESCRIPTION OF THE DRAWINGS

FIG. 1 Conventional fluorescent probes having a xanthene dye as the mother nucleus

FIG. 2 Results of fluorescence imaging using various Si-rhodamines

FIG. 3 Results of x-ray crystal structure analysis of Si-rhodamine having a carboxylic acid

FIG. 4 Schematic diagram of Si-rhodamine localized in cytosol

FIG. 5 Results of fluorescence imaging using Si-rhodamine in which the benzene ring position 2 has been substituted by a methyl group

FIG. 6 Principle of fluorescence control of CaSiR-1 by photoexcitation electron transfer (PeT)

FIG. 7 Results of evaluation of compounds A-C as Ca²⁺ probes

FIG. 8 Results of fluorescence imaging in HeLa cells using CaSiR-2AM

FIG. 9 Visualization of histamine or ATP in HeLa cells utilizing CaSiR-2AM, and results of induced calcium oscillation.

FIG. 10 Visualization of histamine or ATP in HeLa cells utilizing CaSiR-1AM, and results of induced calcium oscillation.

FIG. 11 Results of Ca²⁺ imaging by CaSiR-2AM and CaSiR-1AM in rat brain slices.

FIG. 12 Results of fluorescence imaging by co-staining of CaSiR-1 in rat brain slices.

FIG. 13 Cytosolic, lysosomal, and whole cell fluorescent traces in rat brain slices cultured with CaSiR-1

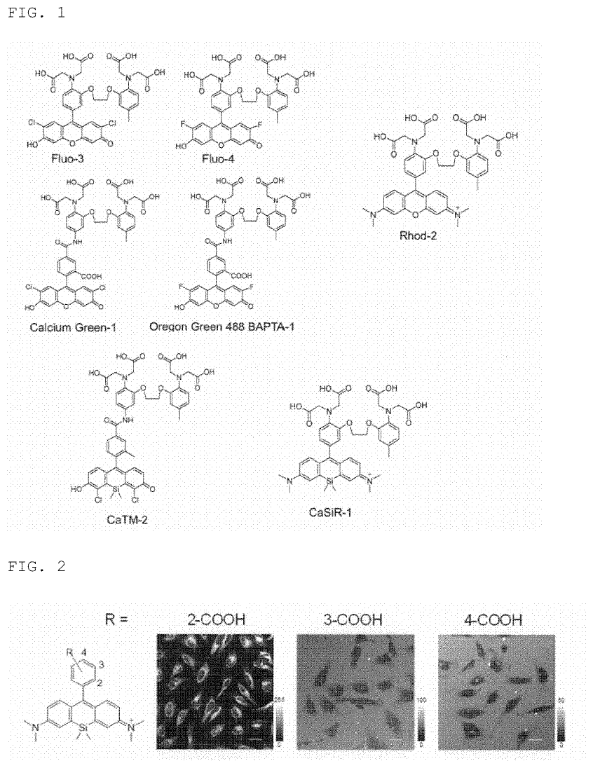

### BEST MODE FOR CARRYING OUT THE INVENTION

In the present specification, an “alkyl group” or alkyl moiety of a substituent including an alkyl moiety (such as an alkoxy group), when not mentioned in particular, means a C1-6, preferably C1-4, or more preferably C1-3 alkyl group that is linear, branched, cyclic or a combination of these forms. More specific examples include a methyl group, ethyl group, n-propyl group, isopropyl group, cyclopropy group, n-butyl group, sec-butyl group, isobutyl group, tert-butyl group, cyclopropylmethyl group, n-pentyl group, n-hexyl group, etc., as alkyl groups.

When “halogen atom” is stated in the present specification, it may be any of a fluorine atom, chlorine atom, bromine atom, or iodine atom, preferably a fluorine atom, chlorine atom, or bromine atom.

One embodiment of the present invention is a compound represented by the following general formula (1), or a salt thereof.

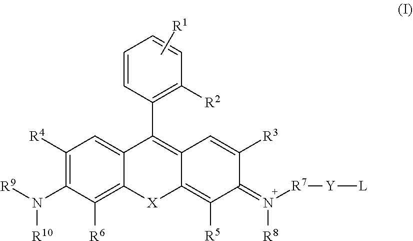

In general formula (I), R¹ represents a hydrogen atom or represents from one to four of the same or different monovalent substituents present on the benzene ring. R¹ may be the same or different.

When R¹ represents a monovalent substituent present on the benzene ring, about one or two of the same or different substituents are preferably present on the benzene ring.

When R¹ represents one or more monovalent substituents, the substituents can substitute any position on the benzene ring. Preferably, all R¹ are hydrogen atoms, or one R¹ is a monovalent substituent and the other R¹ are hydrogen atoms.

The type of monovalent substituent represented by R¹ is not particularly limited, but R¹ is selected, for example, from the group consisting of C1-6 alkyl groups, C1-6 alkenyl groups, C1-6 alkynyl groups, C1-6 alkoxy groups, hydroxyl groups, carboxy groups, sulfonyl groups, alkoxycarbonyl groups, halogen atoms, amino groups, and substituents that act as a capturing group on the substance to be measured.

One or more halogen atoms, carboxy groups, sulfonyl groups, hydroxyl groups, amino groups, alkoxy groups, etc., may be present in alkyl groups represented by R¹.

Alkyl groups represented by R¹ may be alkyl halide groups, hydroxyalkyl groups, carboxyalkyl groups, aminoalkyl groups, etc.

One or two alkyl groups may be present in amino groups represented by R¹; amino groups represented by R¹ may be monoalkylamino groups or dialkylamino groups; when the alkoxy groups represented by R¹ have substituents, the alkoxy groups may be carboxy-substituted alkoxy groups or alkoxycarbonyl-substituted alkoxy groups (for example, a 4-carboxybutoxy group, 4-acetaxymethyloxycarbonylbutoxy group, etc.).

The type of substance to be measured of the capturing group of R¹ is not particularly limited and, for example, may be any of a proton, metal ion (for example, a sodium ion, lithium ion, or other such alkali metal ion; calcium ion or other such alkaline earth metal ion; magnesium ion; zinc ion; etc.), nonmetal ion (carbonate ion, hydroxide ion, etc.), low-oxygen environment, active oxygen species (for example, a hydroxyl radical, peroxynitrite, hypochlorous acid, hydrogen peroxide, etc.), nitrogen monoxide, hydrogen peroxide, singlet oxygen, a pH environment, an enzyme, etc.

The capturing group of R¹ is preferably a capturing group for capturing a proton, metal ion, lowoxygen environment, active oxygen species, nitrogen monoxide, hydrogen peroxide, singlet oxygen, or pH environment.

Here, the metal ion is selected from a zinc ion, magnesium ion, sodium ion, potassium ion, or calcium ion. Preferably, the metal ion is a calcium ion.

Specific types of capturing groups of R¹ are the same as the substituents that act as a capturing group on a substance to be measured of Z described below.

The capturing group of R¹ may be the same as or different from the capturing group of L. Also, the substance to be measured on which the capturing group of R¹ acts may be the same as or different from the substance to be measured on which the capturing group of L acts.

In one aspect of the present invention, the capturing group of R¹ is a calcium ion capturing group. Also, in one aspect of the present invention, the capturing group of R¹ is a calcium ion capturing group represented by formula (1) or (2) described below.

Also, in one aspect of the present invention the capturing groups of R¹ and L are calcium ion capturing groups. Also, in one aspect of the present invention, the capturing groups of R¹ and L are calcium ion capturing groups represented by formula (1) or (2) described below.

In one preferred aspect, R are monovalent substituent such as C1-6 alkyl groups, etc., and said substituents are present at from positions 3 to 6 on the benzene ring.

In one preferred aspect of the present invention, R¹ are all hydrogen atoms.

In the present invention, in general formula (I), R² is an anionic functional group, a C1-10 alkyl group, or a C1-10 alkoxy group, preferably an anionic functional group.

Although not intended to be bound by theory, by eliminating the cationicity of the rhodamine by introducing an anionic functional group into the benzene ring of the xanthene skeleton, accumulation in specific intracellular organelles such as the mitochondria derived from cationicity can be suppressed and more can remain in the cytoplasm.

Also, when an anionic functional group such as a carboxylic acid which is a water-soluble functional group is introduced into the molecular skeleton, the cell membrane permeability generally decreases, but rhodamine with an anionic functional group such as a carboxy group introduced at position 2 of the benzene ring of the xanthene skeleton can exhibit high cell membrane permeability without being strongly retained in specific organelles.

The anionic functional group of R² is selected from a hydroxyl group, carboxy group, C1-10 hydroxyalkyl group, C1-10 alkyl group having a carboxy group, or C1-10 alkoxy uroup having a carboxy group.

The anionic functional group is preferably a hydroxyl group, carboxy group, sulfo group, or C1-10 alkyl group having a carboxy group, more preferably a carboxyl group.

Examples of C1-10 alkyl groups of R² include a methyl group, ethyl group, etc.; examples of C1-10 alkoxy groups include a methoxy group, ethoxy group, etc.

In general formula (I), R³ and R⁴ each independently represent a hydrogen atom, a C1-6 alkyl group, or a halogen atom.

When R³ and R⁴ represent alkyl groups, one or more halogen atoms, carboxy groups, sulfonyl groups, hydroxyl groups, amino group, alkoxy groups, etc., may be present in the alkyl group; for example, alkyl groups represented by R³ and R⁴ may be alkyl halide groups, hydroxyalkyl groups, carboxyalkyl groups, etc. R³ and R⁴ each independently are preferably a hydrogen atom or a halogen atom. It is more preferred when both R³ and R⁴ are hydrogen atoms or when both R³ and R⁴ are fluorine atoms or chlorine atoms.

In general formula (I), R⁵ and R⁶ each independently represent a hydrogen atom, a C1-6 alkyl group, or a halogen atom, the same as was explained for R³ and R⁴. R⁵ and R⁶ are preferably both hydrogen atoms, are both chlorine atoms, or are both fluorine atoms.

In general formula (I), X is SiR¹¹R¹², GeR¹¹R¹², SnR¹¹R¹², CR¹¹R¹², SO₂, or POR¹³. X is preferably SiR¹¹R¹² or GeR¹¹R¹², more preferably SiR¹¹R¹².

R¹¹ and R¹² each independently represent a C1-6 alkyl group or an aryl group. R¹¹ and R¹² each independently are preferably a C1-3 alkyl group, and R¹¹ and R¹² are both more preferably methyl groups. One or more halogen atoms, carboxy groups, sulfonyl groups, hydroxyl groups, amino groups, alkoxy groups, etc., may be present in alkyl groups represented by R¹¹ and R¹²; for example, alky groups represented by R¹¹ and R¹² may be alkyl halide groups, hydroxyalkyl groups, carboxyalkyl groups, etc. When R¹¹ and R¹² represent aryl groups, the aryl groups may be monocyclic aromatic groups or condensed aromatic groups; and the aryl ring may include one or more ring member heteroatoms (for example, a nitrogen atom, oxygen atom, or sulfur atom). A phenyl group is preferred as the aryl group. One or more substituents may be present on the aryl ring. For example, one or more halogen atoms, carboxy groups, sulfonyl groups, hydroxyl groups, amino groups, alkoxy groups, etc., may be present as substituents.

R¹³ represents a C1-6 alkyl group or an optionally substituted phenyl group. Examples of phenyl group substituents include a methyl group, hydroxy group, methoxy group, etc.

R¹³ is preferably a methyl group or phenyl group in terms of the ease of synthesis. Also, R¹³ being a methyl group is more preferred for the higher water solubility.

In general formula (1), R⁸ represents a hydrogen atom or a C1-6 alkyl group.

Also, R⁸, together with may form a five- to seven-membered heterocyclyl or heteroaryl including the nitrogen atoms to which R⁸ is bonded, may also contain from one to three heteroatoms selected from the group consisting of an oxygen atom, nitrogen atom, and sulfur atom as ring members, and the heterocyclyl or heteroaryl may also be substituted by a C1-6 alkyl, C2-6 alkenyl, or C2-6 alkynyl, C6-10 aralkyl group (such as a benzyl group, phenethyl group, etc.), or C6-10 alkyl-substituted alkenyl group. Examples of the heterocyclyl or heteroaryl formed in this way include, but are not limited to, pyrrolidine, piperidine, hexamethyleneimine, pyrrole, imidazole, pyrazole, oxazole, thiazole, etc.

In one preferred aspect of the present invention, R³ is selected from a methyl group or ethyl group.

R⁹ and R¹⁰ each independently represent a hydrogen atom or a C1-6 alkyl group.

Also, R⁹ and R¹⁰ together may form a four- to seven-membered heterocyclyl containing a nitrogen atom to which R⁹ and R¹⁰ are bonded. Examples of the heterocyclyl include azetidine, pyrrolidine, etc., and these heterocyclyls may be substituted by substituents such as C1-6 alkyl groups.

Also, R⁹ or R¹⁰, or both R⁹ and R¹⁰, together with R⁴, R⁶, respectively, may form a five- to seven-membered heterocyclyl or heteroaryl containing a nitrogen atom to which R⁹, R¹⁰ are bonded, may also contain from one to three heteroatoms selected from the group consisting of an oxygen atom, nitrogen atom, and sulfur atom as ring members, and the heterocyclyl or heteroaryl may also be substituted by a C1-6 alkyl, C2-6 alkenyl, or C2-6 alkynyl, C6-10 aralkyl group (such as a benzyl group, phenethyl group, etc.), or C6-10 alkyl-substituted alkenyl group. Examples of the heterocyclyl or heteroaryl formed in this way include, but are not limited to, pyrrolidine, piperidine, hexamethyleneimine, pyrrole, imidazole, pyrazole, oxazole, thiazole, etc.

In one preferred aspect of the present invention, R⁹ and R¹⁰ each independently are selected from a methyl group or ethyl group.

In the present invention, in general formula (I), it is important that L, which is a substituent (capturing group) that acts as a capturing group on a substance to be measured, have a structure bonded via a linker (R⁷—Y) extended via a nitrogen atom of the xanthene ring.

Although not intended to be bound by theory, a capturing group can also be introduced into the benzene ring bonded to the xanthene ring, but the shorter the distance between the BAPTA site used suitably in the capturing group of a Ca²⁺ probe and the xanthene ring (the smaller the number of bonds between the BAPTA structure and the xanthene ring), the higher an S/N ratio can be exhibited by better quenching by PeT (photoexcitation electron transfer) in the absence of Ca²⁺.

In general formula (I) R⁷ represents a C1-6 alkylene group, and the alkylene group may have substituents (for example, a hydroxy group, methoxy group). R⁷ is preferably a methylene group or ethylene group.

In general formula (I), Y, when present, represents a spacer that bonds L and the benzene ring. Amides (—CO—NH—), esters (—COO—), thiourea, etc., can be used as spacers, but amides or esters are preferred, and amides are more preferred.

In general formula (I), L represents a substituent that acts as a capturing group on a substance to be measured.

Types of substances to be measured include, but are not limited to, a proton, metal ion (for example, a sodium ion, lithium ion, or other such alkali metal ion; calcium ion or other such alkaline earth metal ion; magnesium ion; zinc ion; etc.), nonmetal ion (carbonate ion, hydroxide ion, etc.), low-oxygen environment, active oxygen species (for example, a hydroxyl radical, peroxynitrite, hypochlorous acid, hydrogen peroxide, etc.), nitrogen monoxide, hydrogen peroxide, singlet oxygen, a pH environment, an enzyme, etc. In the present invention, a proton, metal ion, low-oxygen environment, active oxygen species, nitrogen monoxide, hydrogen peroxide, singlet oxygen, or pH environment are preferred, and a metal ion is more preferred.

The metal ion is preferably selected from a zinc ion, magnesium ion, sodium ion, potassium ion, or calcium ion, and is preferably a calcium ion.

Various capturing groups that specifically capture a substance to be measured have been proposed and can be selected as is suitable in accordance with the type of substance to be measured. For example, capturing groups described in JPH10-226688A, International Publication WO99/51586, JP2000-239272A, International Publication WO01/62755, etc., as well as the catalog of Molecular Probes, Inc. (Molecular Probes Handbook 11th Edition) Chapter 10 (Enzyme substrates and analysis), Chapter 17 (Signaling probes), Chapter 18 (Nitrogen monoxide-containing active oxygen species probes), Chapter 19 (Calcium ion, magnesium ion, zinc ion, and other metal ion indicators), Chapter 20 (pH indicators), and Chapter 21 (Sodium ion, potassium ion, chlorine ion, and other ions) can be used. Capturing groups, however, are not limited to those described in the above publications.

In the present specification, the term “capturing” includes cases in which the capturing group does not cause any substantial chemical change as in capture by chelation, etc., of a metal ion, etc., as well as when the chemical structure is changed by a chemical reaction with the substance to be measured and when the capturing group is cleaved and eliminated by contact with an enzyme. The term must be interpreted in the broadest sense and must not be interpreted restrictively in any sense.

Examples of capturing groups include capturing groups represented by (A) to (K) below, but capturing groups that can be used in the present invention are not restricted to these examples.

### (A) Zinc Ion Capturing Groups

### (A-1)

A capturing group represented by

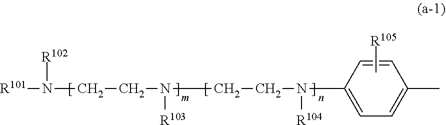

(in the formula, R¹⁰¹, R¹⁰², R¹⁰³, and R¹⁰⁴ each independently represent a hydrogen atom, alkyl group, 2-pyridylmethyl group, 2-pyridylethyl group, 2-methyl-6-pyridylmethyl group, or 2-methyl-6-pyridylethyl group, but at least one selected from the group consisting of R¹⁰¹, R¹⁰², R¹⁰³, and R¹⁰⁴ represents a group selected from the group consisting of a 2-pyridylmethyl group, 2-pyridylethyl group, 2-methyl-6-pyridylmethyl group, and 2-methyl-6-pyridylethyl group; R¹⁰⁵ is a hydrogen atom or represents one to four of the same or different monovalent substituents present on the benzene ring; m and n each independently represent 0 or 1, but m and n are not simultaneously 0).

The above capturing groups are disclosed in Japanese Patent. No. 4402191 and J. Am. Chem. Soc., 127, pp. 10197-10204, 2005.

Suitable examples of the above capturing group include capturing groups represented by the following formula.

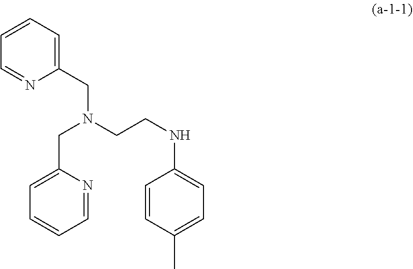 [SMILES: Cc1ccc(NCCN(Cc2ccccn2)Cc2ccccn2)cc1]

Also, these capturing groups may bond to the benzene ring via a spacer such as —CO—NH— as described below. For example, a capturing group of formula (a-1-1) is represented by the following formula when bonded to a benzene ring via a —CO—NH— spacer.

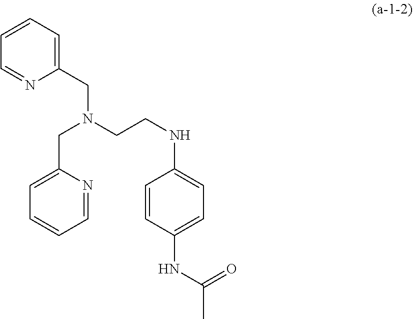 [SMILES: CC(=O)Nc1ccc(NCCN(Cc2ccccn2)Cc2ccccn2)cc1]

### (A-2)

A capturing group represented by

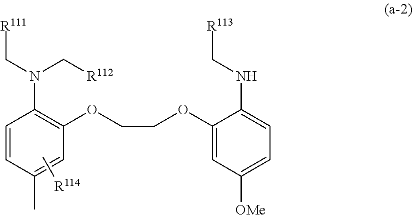

(in the formula R¹¹¹, R¹¹², and R¹³³ each independently represent a carboxy group and a salt thereof, R¹¹⁴ is a hydrogen atom or represents one to three of the same or different monovalent substituents present on the benzene ring).

The above capturing groups are disclosed in J. Am. Chem. Soc., 124, pp. 776-778, 2002.

### (A-3)

A capturing group represented by

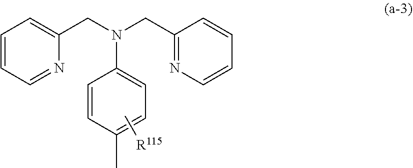

(in the formula, R¹¹⁵ is a hydrogen atom or represents one to four of the same or different monovalent substituents present on the benzene ring.

The above capturing groups are described in U.S. Pat. No. 5,648,270.

### (A-4)

A capturing group represented by

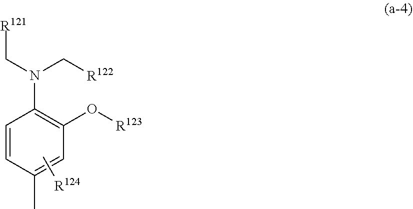

(in the formula, R¹²¹ and R¹²² each independently represent a carboxy group and a salt thereof; R¹²³ represents a C1-6 alkyl group; R¹²⁴ represents one to three of the same or different monovalent substituents including a hydrogen atom on the benzene zing).

The above capturing groups are disclosed in Cell Calcium, 31, pp. 245-251, 2002.

### (A-5)

A capturing group represented by

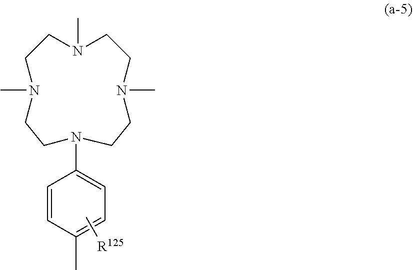

(in the formula, R¹²⁵ is a hydrogen atom or represents one to four of the same or different monovalent substituents including a hydrogen atom present on the benzene ring).

The above capturing groups are disclosed in JP 2000-239272A.

### (B) Nitrogen Monoxide Capturing Groups

A capturing group represented by

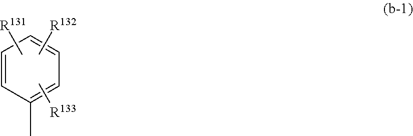

(in the formula, R¹³¹ and R¹³² represent substituents substituted at adjacent positions on the benzene ring and each independently represent an amino group or a C1-6 alkyl mono-substituted amino croup, but R¹³¹ and R¹³² do not simultaneously represent C1-6 alkyl mono-substituted amino groups; R¹³³ is a hydrogen atom or represents one to three of the same or different monovalent substituents present on the benzene ring).

The above capturing groups are disclosed in Japanese Patent No. 3200024, U.S. Pat. No. 6,441,197, U.S. Pat. No. 675,623, and Japanese Patent. No. 3967943.

### (C) Active Oxygen Species Capturing Groups

A capturing group represented by

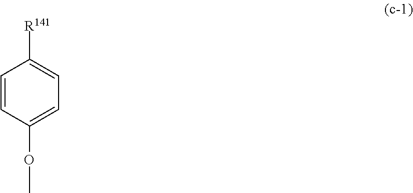

(in the formula, R¹⁴¹ represents an amino group or a hydroxy group).

The above capturing groups are disclosed in International Publication WO2001/064664.

### (D) Low-Oxygen Environment Capturing Groups

### (D-1)

A capturing group represented by

—CO—N(R¹⁵¹)—Y¹—N(R¹⁵²)—X¹—(X²)ᵣ-p-C₆H₄—N═N—Ar—R¹⁶³ (d-1)

[in the formula, R¹⁵¹ and R¹⁵² each independently represent a hydrogen atom or a C1-6 alkyl group, R¹⁵¹ and R¹⁵² may bond to each other to become a C2-6 alkylene group; Y¹ represents a C1-6 alkylene group; X¹ represents a single bond, —CO—, or —SO₂—; X² represents —O—Y²—N(R¹⁵⁴)— (in the formula, Y² represents a C1-6 alkylene group, R¹⁵⁴ represents a hydrogen atom or a C1-6 alkyl group); r represents 0 or 1; p-C₆H₄— represents a p-phenylene group; Ar represents an aryldiyl group; R¹⁵³ represents a monoalkylamino group or a dialkylamino group].

The above capturing groups are disclosed in International Publication WO2010/026743.

### (D-2)

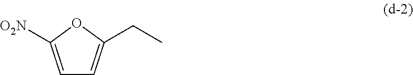

The above capturing groups are disclosed in JP 2009-275006A.

### (E) Hydrogen Peroxide Capturing Groups

A capturing group represented by

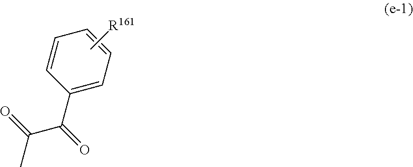

(in the formula R¹⁶¹ represents one or more electron-withdrawing substituents present on the benzene ring).

The above capturing groups are disclosed in International Publication WO2009/110487.

### (F) Singlet Oxygen Capturing Groups

A capturing group represented by

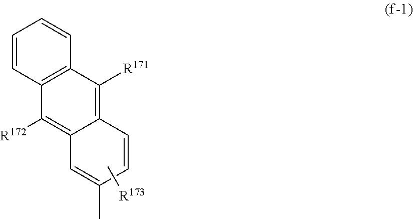

(in the formula, R¹⁷¹ and R¹⁷² each independently represent a C1-4 alkyl group or an aryl group; R¹⁷³ is a hydrogen atom or represents one to three of the same or different monovalent substituents present on the benzene ring).

The above capturing groups are disclosed in Japanese Patent No. 4373608 and International Publication WO2002/018362.

### (G) pH Environment Capturing Groups

A capturing group represented by

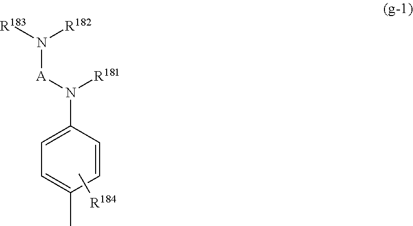

(in the formula, R¹⁸¹, R¹⁸², R¹⁸³ each independently represent a hydrogen atom, an optionally substituted C1-6 alkyl group, or an optionally substituted aryl group, or R¹⁸¹ and R¹⁸² bond to represent a C1-3 alkylene group, or R¹⁸¹ and R¹⁸³ bond to represent a C1-3 alkylene group; A represents an optionally substituted C1-3 alkylene group; R¹⁸⁴ is a hydrogen atom or represents one to four of the same or different monovalent substituents present on the benzene ring).

The above capturing groups are disclosed in International Publication WO2008/099914 and International Publication WO2008/059910.

### (H) Magnesium Ion Capturing Groups

A capturing group represented by

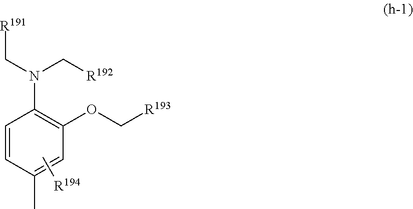

(in the formula, R¹⁹¹, R¹⁹², and R¹⁹³ each independently represent a carboxy group and a salt thereof; R¹⁹⁴ is a hydrogen atom or represents one to three of the same or different monovalent substituents present on the benzene ring).

The above capturing groups are disclosed in the American Journal of Physiology, 256, C540-548, 1989.

### (I) Sodium Ion and Potassium Ion Capturing Groups

A capturing group represented by

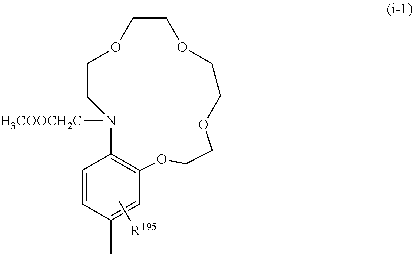

(in the formula, R¹⁹⁵ is a hydrogen atom or represents one to three of the same or different monovalent substituents present on the benzene ring).

The above capturing groups are disclosed in Bioorg. Med. Chem. Lett., 15, pp. 1851-1855, 2005.

### (J) Calcium Ion Capturing Groups

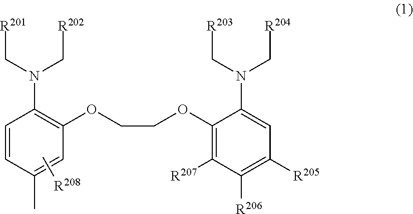

In formula (1), R²⁰¹, R²⁰², R²⁰³, and R²⁰⁴ each independently represent a carboxy group, an alkyl group having a carboxy group, an ester group, an optionally substituted alkyl ester group, or a salt thereof.

R²⁰⁵, R²⁰⁶, and R²⁰⁷ each independently represent a hydrogen atom, a halogen atom (fluorine atom, chlorine atom, and bromine atom), a C1-6 alkyl group, a methoxy group, or a nitro group.

R²⁰⁸ is a hydrogen atom or represents one to three of the same or different monovalent substituents present on the benzene ring.

In a preferred embodiment of the present invention, is general formula (I), L is a calcium ion capturing group represented by the above formula (1).

A capturing group having a BAPTA (1,2-bis(o-aminophenoxy)ethane-N,N,N′,N′ -tetraacetic acid) structure is preferred as a calcium ion capturing group L.

Alternatively,

a capturing group represented by the following formula (2) is preferred as a calcium ion capturing group L.

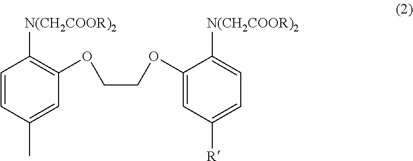

In formula (2), R is hydrogen or —CH₂OCOCH₃, and each R may be the same or different. R′ is a methyl group, methoxy group, or fluorine atom.

### (K) Enzyme Capturing Groups

Examples of enzymes can include reductases, oxidases, hydrolases, etc. For example, β-lactamase, cytochrome P450 oxidase, β-galactosidase, β-glucosidase, β-glucuronidase, β-hexosaminase, lactase, alkaline phosphatase, matrix metalloprotease, glutamyl transferase, etc., can be given as examples of enzymes useful in the diagnosis of infection, cancer, etc, but are not limited thereto. Hydrolases are especially preferred among enzymes. Typical examples of hydrolases include β-galactosidase, β-lactamase, alkaline phosphatase, matrix metalioprotease, glutamyl transferase, etc., but hydrolases are not limited to the above examples.

When a hydrolase is used as the substance to be measured, compounds and functional groups to serve as a specific substrate of the enzyme are selected to make it possible to design a compound of general formula (I) that gives a compound in which L (and R¹) is a hydrogen atom upon hydrolysis by the enzyme. For example, when a sugar hydrolase is used as the substance to be measured, a residue of a sugar compound that serves as a substrate of that enzyme can be used as L¹ (and R¹). Functional groups such as hydroxyl groups and amino groups of the sugar compound may be protected by appropriate protecting groups as needed. Compounds having such protecting groups are also all encompassed within the scope of the present invention.

When a peptidase or protease is used as the substance to be measured, acyl residues derived from 20 types of L-amino acids that construct a protein including an amino acid residue (the amino acid residue represents a group in which one hydrogen atom has been removed from an amino group or carboxy group of the amino acid) substituted by a substituent described as a fluorescent probe of (a)-(g) and GGT in the present specification or a compound of (1) to (7) described in [Chemical formula 4] on page 12 of International Publication WO2010/095450 (the above amino acid residue may bond to Y or R⁷ to which L of general formula (I) of the present specification is bonded) can be given as examples of monovalent substituents cleaved by contact with an enzyme. Also, when a lactamase is used as the substance to be measured, examples include substituents described in formula (h) in the present specification; when a sugar hydrolase is used, examples include a galactosyl group, glucosyl group, and glucuronosyl group; and when a glucuronosyltransferase is used, examples include monovalent substituents cleaved by contact of a hydroxyl group, amino group, carboxy group, or thiol group with an enzyme.

When glutathione is used as the substance to be measured, examples of monovalent substituents cleaved by contact with the substance to be measured include substituents described in formula (i) of the present specification.

Examples of substances to be measured include enzymes (peptidases, proteases, lactamases, sugar hydrolases, transferases, oxidoreductases, etc.) and glutathione. For example, peptidases, proteases, or lactamases are preferred as enzymes.

The type of peptidase or protease is not particularly limited as long as the acyl group can be hydrolyzed in a compound of the present invention represented by the above general formula (I) in which I (and R¹) is an acyl group; the peptidase may be either an endopeptidase or an exopeptidase, and the protease may be either an endoprotease or an exoprotease. For example, to measure a peptidase or protease having a specific amino acid or peptide as the substrate, an acyl residue derived from said amino acid or peptide can be used in L (and R¹), and the specific peptidase or protease can be measured specifically by using a compound designed in this way (the acyl residue derived from the amino acid or peptide represents a partial structure remaining after removing a hydroxyl group from a carboxy group of the amino acid). From this viewpoint, it is preferable to use an acyl residue derived from an amino acid or derived from a peptide that can be hydrolyzed by the peptidase or protease as L and (R¹) as a fluorescent probe for the peptidase or protease, and, for example, acyl residues derived from the 20 types of L-amino acids that construct the protein or acyl residues derived from selenocysteine, pyrolysin, cystine, hydroxyproline, hydroxylysine, thyroxine, O-phosphoserine, desmosine, β-alanine, sarcosine, ornithine, creatine, γ-aminobutyric acid, opine, etc., can be used.

When the peptidase is an LAP (leucine aminopeptidase), examples of suitable R¹¹ (R⁹, R¹⁰, or R¹³) include the following substituent.

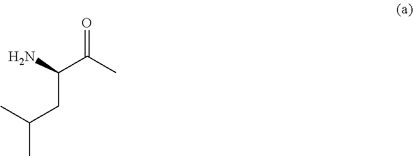

When the peptidase is a GGT (γ-glutamyl transpeptidase), examples of suitable R¹¹ include the following substituent. For example, if a compound having the following substituent as R¹¹ is used instead of γGlu-RhoHM according to the method described in International Publication WO2011/087000, cancer cells and cancer tissues can be measured specifically, and the probe can be utilized as a cancer diagnostic.

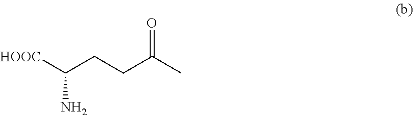

When the protease is caspase-3, examples of suitable L (and R¹) include the following substituents.

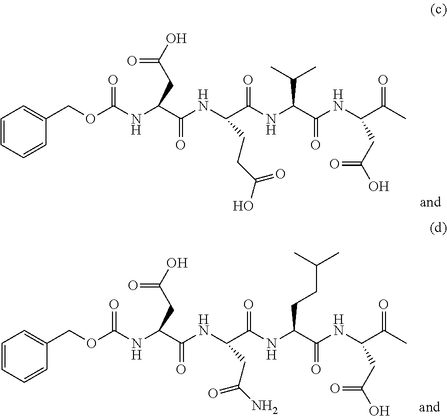 [SMILES: CC(=O)[C@H](CC(=O)O)NC(=O)[C@@H](NC(=O)[C@H](CCC(=O)O)NC(=O)[C@H](CC(=O)O)NC(=O)OCc1ccccc1)C(C)C ; CC(=O)[C@H](CC(=O)O)NC(=O)[C@H](CCC(C)C)NC(=O)[C@H](CC(N)=O)NC(=O)[C@H](CC(=O)O)NC(=O)OCc1ccccc1]

When the protease is calpain, examples of suitable L (R¹) include the following substituents.

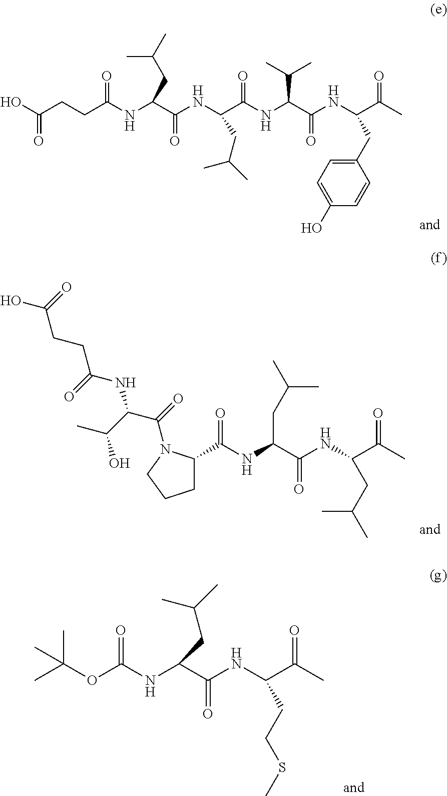

When the lactamase is a β-lactamase, examples of suitable L (and R¹) include the following substituent.

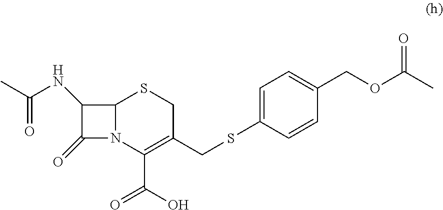 [SMILES: CC(=O)NC1C(=O)N2C(C(=O)O)=C(CSc3ccc(COC(C)=O)cc3)CSC12]

When the substance to be measured that is cleaved by contact is glutathione, examples of suitable L (R¹) include the following substituent.

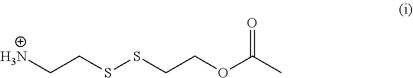

In one preferred embodiment of the present invention, in general formula (I), R² is a carboxy group.

In one preferred embodiment of the present invention, in general formula (I), R² is a carboxy group, R⁷ is selected from a methylene group or an ethylene group and R⁸ is selected from a methyl group or an ethyl group.

In one preferred embodiment of the present invention, in general formula (I), R² is a carboxy group, R⁷ is selected from a methylene group or an ethylene group, R⁸ is selected from a methyl group or an ethyl group, and R⁹ and R¹⁰ are each independently selected from a methyl group or an ethyl group.

In one preferred embodiment of the present invention, in general formula (I), R² is a carboy group, R⁷ is a methylene group, and R⁸, R⁹, and R¹⁰ are all methyl groups.

Non-limiting examples of compounds of general formula (I) of the present invention include the following compounds.

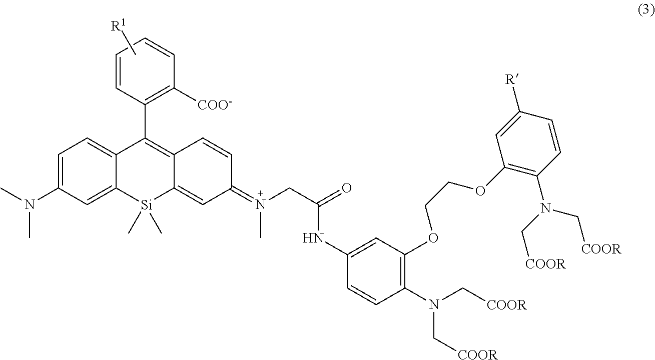

In formula (3), R is hydrogen or —CH₂OCOCH₃, and each R may be the same or different. Also, R′ is a methyl group or a fluorine atom, and R¹ is as defined in general formula (I).

In a preferred aspect of compounds of formula (3), R¹ are monovalent substituents such as C1-6 alkyl groups, etc., and said substituents are present at from positions 3 to 6 on the benzene ring.

In a preferred aspect of compounds of formula (3), R¹ are all hydrogen atoms.

Compounds of general formula (I) and (3) of the present invention can be present as acid addition salts or base addition salts. Examples of acid addition salts include hydrochlorides, sulfates, nitrates, and other such mineral acid salts, or methanesulfonates, p-toluenesulfonates, oxalates, citrates, tartrates, and other such organic acid salts; examples of base addition salts include sodium salts, potassium salts, calcium salts, magnesium salts, and other such metal salts, ammonium salts, or triethylamine salts and other such organic amine salts. In addition to these, there are also cases in which salts form with an amino acid such as glycine. Compounds or salts thereof of the present invention can also exist as hydrates or solvates, but these substances are also within the scope of the present invention.

Compounds of general formula (I) and (3) of the present invention sometimes have one or more asymmetrical carbons, depending on the types of substituents. In addition to optical isomers based on one or more asymmetrical carbons and stereoisomers such as diastereomers based on two or more asymmetrical carbons, any mixtures of stereoisomers, racemates, etc., are all encompassed within the scope of the present invention.

Methods for producing representative compounds of compounds represented by general formula (I) of the present invention are specifically shown in the examples in the present specification. Therefore, one skilled in the art can produce compounds of the present invention represented by general formula (I) by appropriately selecting the reaction raw materials, reaction conditions, reaction reagents, etc. based on these explanations and modifying or changing these methods as needed.

One more embodiment of the present invention is a fluorescent probe that includes any compound of general formula (I) or salt thereof.

Also, one more embodiment of the present invention is a method for measuring a substance to be measured, wherein the method includes (a) a step for bringing the compound represented by general formula (I) or a salt thereof into contact with a substance to be measured and (b) a step for measuring the fluorescence intensity of the compound after capture of the substance to be measured generated in step (a).

In the method of the present invention, the substance to be measured is preferably a calcium ion.

### EXAMPLES

The present invention is explained below through examples, but the present invention is not limited to these examples.

### 1. Development of Rhodamines that Accumulate in the Cytoplasm

Rhodamane dyes generally exhibit localization to organelles such as the mitochondria due to the cation of their xanthene ring. Therefore, the present inventors first studied the possibility of developing rhodamines that accumulate in the cytoplasm and have near-infrared fluorescence by controlling this localization through structural modification.

A method of introducing an anionic functional group such as a carboxylic acid into the structure was considered as a molecular modification to eliminate the cationicity of the rhodamine and make the net charge 0. By eliminating the cationicity of the rhodamine by introducing an anionic functional group in this way, it was thought that the accumulation in intracellular organelles such as the mitochondria derived from cationcity would be suppressed and it would be possible to develop rhodamines of Si, etc., that accumulate more in the cytoplasm. However, on the other hand, introducing an anionic functional group such as a carboxylic acid which is a water-soluble functional group into the molecular skeleton is known to generally lower the cell membrane permeability.

More detailed studies were therefore carried out by synthesizing Si-rhodamines with carboxylic acids introduced at different positions on the benzene ring to explore benzene ring carboxylic acid positions and the cell membrane permeability thereof. Specifically, derivatives having carboxylic acids introduced at positions 2, 3, or 4 of the benzene ring of Si-rhodamines were synthesized. Fluorescence imaging was also carried out by applying the Si-rhodamines synthesized to HeLa cells. FIG. 2 shows fluorescent images taken without washing away the excess extracellular dye.

For measurements, HeLa cells were incubated with 1 μM of Si-rhodamine having a carboxy group. Ex was 633 nm, and Em was 670-750 nm. The scale bar in FIG. 2 is 30 μm.

As shown in FIG. 2, while virtually no fluorescence from inside the cells was observed with Si-rhodamines having carboxy groups at positions 3 and 4 of the benzene ring (center and right-hand photographs in FIG. 2), strong fluorescence was observed from inside the cells with Si-rhodamine having a carboxy group at position 2 (left-hand photograph in FIG. 2). The fluorescence intensity from inside the cells was also greatly attenuated by washing out the Si-rhodamine having a carboxy group at position 2.

Based on the above results, it was clear that Si-rhodamine having a carboxy group at position 2 of the benzene ring is not retained strongly in specific organelles and exhibits high cell membrane permeability.

The above results were considered as follows. Dyes having a carboxylic acid introduced at position 3 or 4 of the benzene ring of Si-rhodamine had decreased cell membrane permeability and fluorescence was not observed from inside the cells, as is observed in many dyes having anionic functional groups. On the other hand, Si-rhodamine with a carboxylic acid introduced at position 2 of the benzene ring, unlike the two above dyes, accumulated in the cytoplasm due to high membrane permeability, and strong fluorescence was observed from inside the cells. The characteristic behavior of such Si-rhodamine having a carboxylic acid at position 2 of the benzene ring was inferred to be due to nucleophilic attack of position 9 of the xanthene ring by the carboxylic acid of position 2 of the benzene ring in the dye molecule and formation of an intramolecular spiro-cyclized state. In short, fluorescence was thought to be observed from inside the cells due to permeation of the cell membrane by formation of an intramolecular spiro-cyclized state, which is highly liposoluble in comparison to the open-ring state, to permeate the cell membrane which is a liposoluble environment, and reformation of an open-ring state inside the cells.

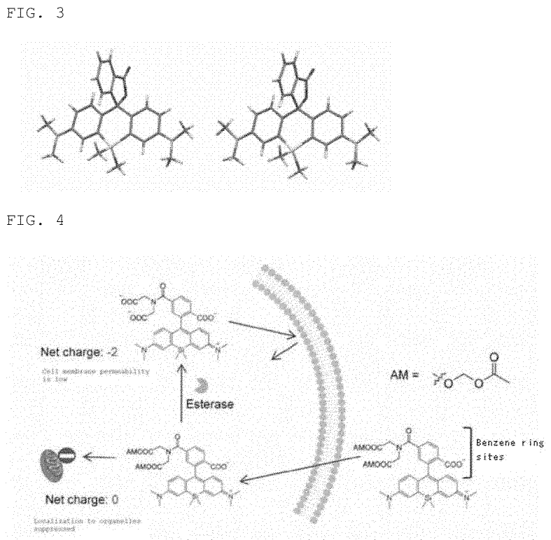

Furthermore, x-ray crystal structural analysis of Si-rhodamine having a carboxylic acid at position 2 of the benzene ring was carried out and the Si-rhodamine was actually confirmed to form an intramolecular spiro-cyclized state as data that support the above behavior (FIG. 3). Specifically, the asymmetrical unit of FIG. 3 contains two crystallographically independent molecules, and 2-COOHSiR650 has an intramolecular spiro-cyclized structure.

Once the above results had been obtained, molecular design of Si-rhodamine that accumulates in the cytoplasm was first carried out as the first step in development of the probe of the present invention. Si-rhodamine having a carboxy group at positions 2 of the benzene ring was used as a fluorophore based on the above results, and an iminodiacetic acid site protected by an acetoxymethyl group (AM group) was introduced to further improve intracellular retention.

After a dye protected by an AM group is introduced into a cell, it is known that the AM group is cleaved by intracellular esterase, decreasing extracellular leakage, and causing the dye to be retained within the cell. It was thought that localization to organelles such as the mitochondria before cleavage of the AM group would be suppressed since the net charge is 0 (see FIG. 4). In addition, Si-rhodamine with position 2 of the benzene ring substituted by a methyl group, which cannot form an intramolecular spiro-cyclized state, was synthesized as a control dye and used in the studies.

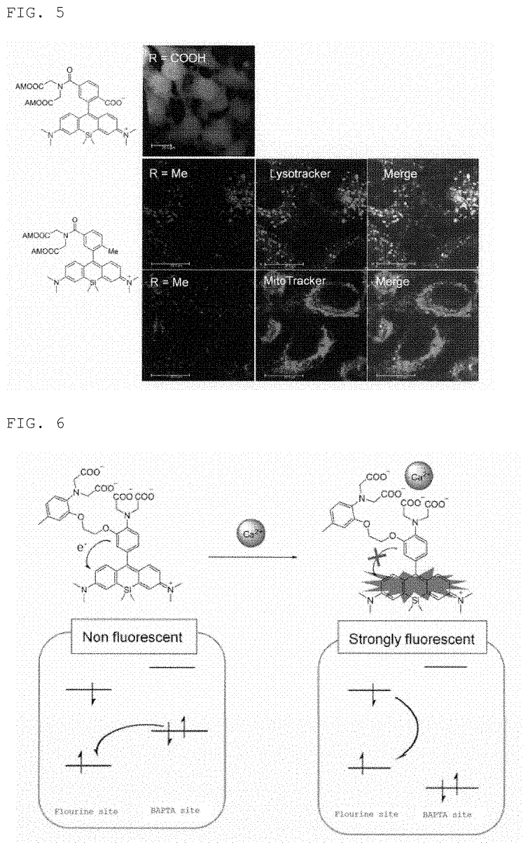

The compounds designed and synthesized were applied to HeLa cells, and fluorescence imaging was conducted (FIG. 5). For the measurements, HeLa cells incubated for one hour with 1 μM of dye (75 nM of LysoTracker of 75 nM of MitoTracker) were used. Ex was 650 nm, and Em was 670-750 nm. The scale bar in FIG. 5 is 20 μm.

Images taken after washing away the excess dye showed. rhodamines with a carboxy group introduced at position 2 of the benzene ring to accumulate in the cytoplasm. On the other hand, the dye having a methyl group at position 2 of the benzene ring exhibited different localization and was understood to localize mainly in the lysosomes as a result of co-staining studies. Therefore, introduction of a carboxy group to the benzene ring site clearly has a major effect on intracellular localization of the rhodamine.

### 2. Molecular Design of a Ca²⁺ Fluorescent Probe Based on Cytoplasm-Accumulating Rhodamine

The above studies succeeded in causing rhodamine to accumulate in the cytoplasm by introducing an iminodiacetic acid structure protected by an Aid group into rhodamines having a carboxylic acid at position 2 of the benzene ring. Next, taking advantage of the knowledge gained by the above studies, molecular design of a Ca²⁺ fluorescent probe was carried out as follows.

The present inventors decided to use photoinduced electron transfer (PeT), which is also applied to existing Ca²⁺ probes, as the fluorescence control principle when detecting Ca²⁺. PeT refers to a phenomenon whereby, when the fluorophore position of a fluorescent probe is excited by excitation light, the fluorescence is quenched by electron transfer from a structure with high electron density near the fluorophore faster than the excited fluorophore returns to the ground state and emits fluorescence. In short, in PeT, the structure with high electron density near the fluorophore during fluorophore excitation becomes an electron donor, and the fluorophore becomes an electron acceptor. PeT is used as the fluorescence control principle of fluorescent probes that capture various physiologically active substance since PeT ceases and the fluorescent property recovers due to lowering of the electron density of the structure that is the electron donor by chemical reaction, etc. In the case of a Cat²⁺ probe using a rhodamine such as CaSiR-1 as the fluorophore, the xanthene ring site serves as the electron acceptor and the aminophenol site of the BAPTA structure serves as the electron donor, but the fluorescent probe becomes basically non-fluorescent due to the occurrence of PeT in the absence of Ca²⁺. On the other hand, the fluorescent property of the probe recovers because the electron density of the aminophenol site of the BAPTA structure is lowered by coordination of the Ca²⁺ ion to the BAPTA structure and electron transfer ceases to occur in the presence of calcium (FIG. 6).

Fluorescence control by PeT can be evaluated by the free energy change ΔG_{eT} of the electron transfer process shown by the Rehm-Weller equation (Reference 3: Johnson I., Spence M. T. Z., Ed. The Molecular Probes Handbook: A Guide to Fluorescent Probes and. Labeling Technologies, 11^{th} Ed. Molecular Probes, Inc. 2010) and the electron transfer rate constant k_{eT} described below by the Marcus equation (Reference 9: Marcus R. A., Annu. Rev. Phys. Chem., 1964, 15, 155-196; Reference 10: Marcus R. A., Sutin, Biochim. biophys. Acta, 1985, 811, 265-322; Reference 11: Marcus R. A., Angew. Chem. Int. Ed., 1993, 32, 1111-1121; Reference 12: De Silva A. P., Gunaratne H. Q., Gunnlaugsson T., Huxley A. J. M., McCoy C. P., Rademacher J. T., Rice T. E., Chem. Rev., 1997, 97, 1515-1566).
Rehm-Weller Equation

ΔG_{eT}−Eₒₓ−E_{red}−ΔE₀₀−C

Eₒₓ: one-electron oxidation potential of electron donor, E_{red}: one-electron reduction potential of electron acceptor, ΔE_{0,0}: excitation energy, C: energy required to pull the radical species generated by excitation out of the Coulombic attraction field
Marcus Equation

$${k}_{eT} = {( \frac{4 {π}^{3}}{{h}^{2} λ {k}_{B} T} )}^{1 / 2} {V}^{2} exp [ - \frac{{( Δ {G}_{eT} + λ )}^{2}}{4 λ {k}_{B} T} ]$$

V: orbit interaction, λ: reorientation energy, k_{B}: Boltzmann's constant, h: Planck's constant, I: temperature

Among the above parameters are parameters involved in the distance between the electron donor and electron acceptor in the electron transfer process. First, V is a parameter involved in the interaction of the electron orbits of the electron donor and electron acceptor, and the value becomes larger as the distance between the two closes. Also, λ is the reorientation energy of the surrounding reaction environment associated with electron transfer and evaluates how much other molecular species such as water come between the electron. donor and electron acceptor in the charge separation state. Furthermore, C is also a parameter that has a value that becomes larger when the electron donor and electron acceptor are adjacent.

Therefore, in developing a new Ca²⁺ probe having near-infrared fluorescence, the following probes having different distances between the BAPTA site and the xanthene ring were designed and synthesized. Compound A, compound B, and compound C shown below have different numbers of bonds of 7, 6, and 5, respectively, between the BAPTA structure and the xanthene ring. It was thought that the smaller the number of bonds, the better quenching by PeT would be in the absence of Ca²⁺.

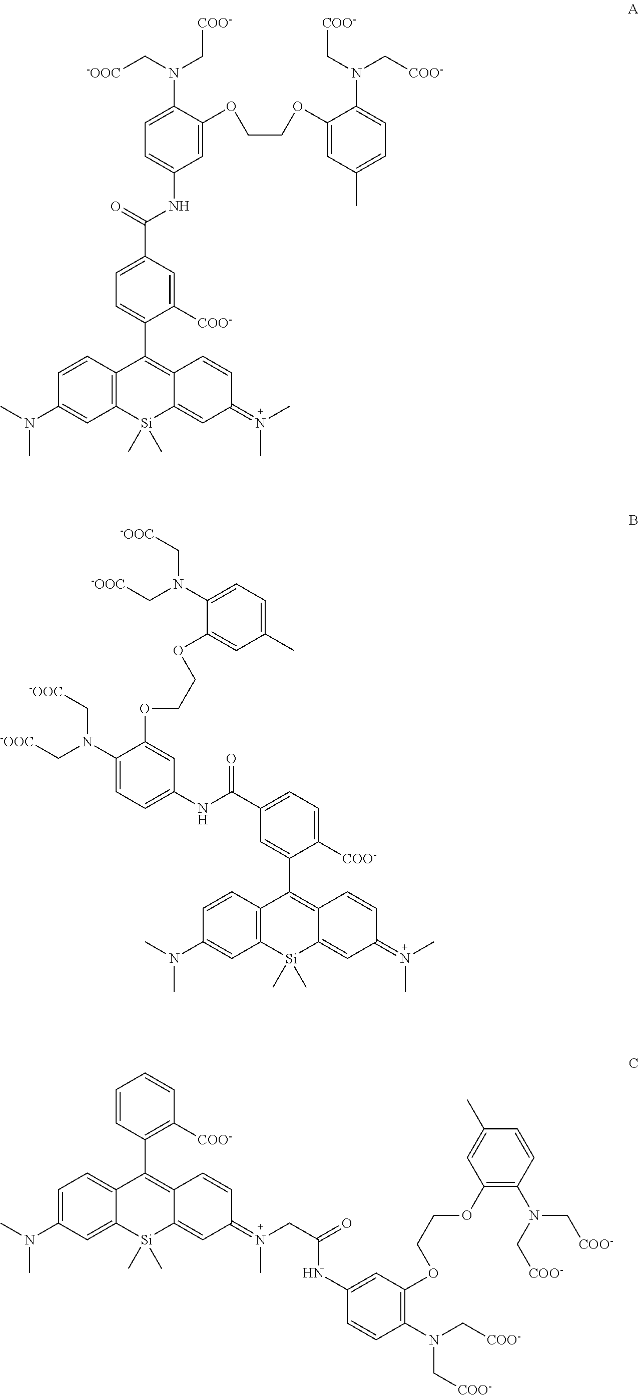

### Synthesis Example 1

### Synthesis of BAPTA

The synthesis intermediate BAPTA used to synthesize compounds A-C was synthesized according to scheme 1 below.

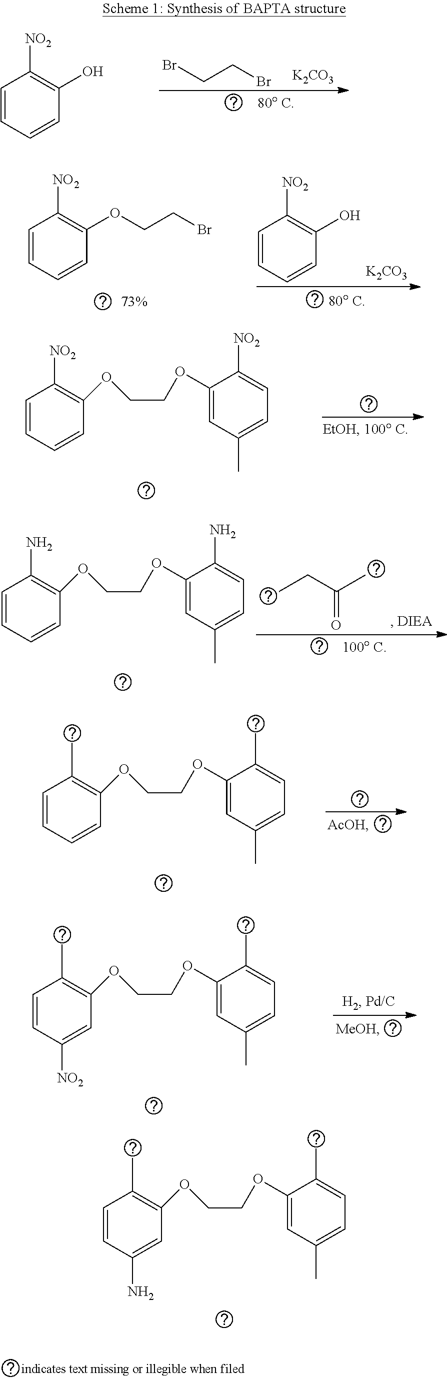

(1) Synthesis of 1-(2-bromoethoxy)-2-nitrobenzene

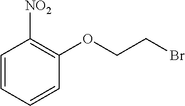 [SMILES: O=[N+]([O-])c1ccccc1OCCBr]

Synthesized according to Reference 1 (Dong X., Yang Y., Sun J., Liu Z., Liu B. F., Chem. Commun., 2009, 26, 3883).

(2) Synthesis of 4-methyl-1-nitro-2-[2-(2-nitrophenoxy)ethoxy]-benzene

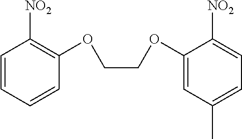 [SMILES: Cc1ccc([N+](=O)[O-])c(OCCOc2ccccc2[N+](=O)[O-])c1]

Synthesized according to Reference 1 above.

(3) Synthesis of 2-[2-(2-aminophenyl)ethoxy]-4-methyl-benzene amine

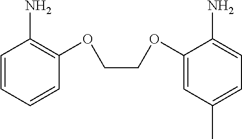 [SMILES: Cc1ccc(N)c(OCCOc2ccccc2N)c1]

Synthesized according to Reference 1 above

(4) Synthesis of 5-methyl BAPTA tetramethyl ester

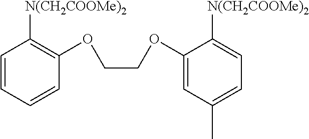

2-[2-(2-aminophenyl)ethoxy]-4-methylbenzene amine (724 mg, 2.81 mmol), methyl bromoacetate (1.54 mL, 16.7 mmol), and DIEA (5.8 mL, 33.3 mmol) were dissolved in MeCN (20 mL) and stirred overnight at 80° C. AcOEt (20 mL) was added; after removing the salt by filtration, the solvent was removed under reduced pressure. The residue was purified by silica gel column chromatography (EtOAc/n-hexane=½), and 5-methyl BAPTA tetramethyl ester was obtained (362 mg, 0.662 mmol, yield 23%).

¹H NMR (400 MHz, CDCl₂): δ=2.26 (s, 3H), 3.56 (s, 6H), 3.58 (s, 6H), 4.12 (s, 4H), 4.16 (s, 4H), 4.27 (s, 4H), 6.67 (d, J=4.9 Hz, 2H), 6.74 (dd, J=4.9 Hz, 3.4 Hz, 1H), 6.81-6.83 (m, 1H), 6.85-6.89 (m, 2H), 6.90-6.93 (m, 1H). ¹³C NMR (100 MHz, CDCl₃): δ=20.9, 51.5, 51.6, 53.3, 53.4, 67.0, 67.1, 113.2, 114.1, 119.0, 119.2, 121.5, 121.7, 122.2, 132.1, 136.8, 139.2, 150.3, 150.4, 172.0, 172.0.

(5) Synthesis of 5-methyl-5′-nitro BAPTA tetramethyl ester

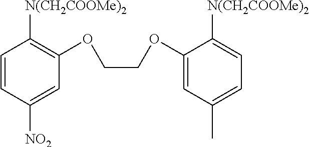

Synthesized according to Reference 2 (Egawa T., Hanaoka K., Koide Y., Ujita S., Takahashi N., Ikegaya Y., Matsuki N., Tera T., Ueno T., Komatsu T., Nagano T., J. Am. Chem. Soc., 2011, 133, 14157-14159).

(6) Synthesis of 5-amino-5′-methyl BAPTA tetramethyl ester

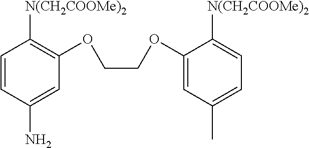

Synthesized according to Reference 2 above.

### Synthesis Example 2 (Reference Example)

Compound A was synthesized according to scheme 2 below.

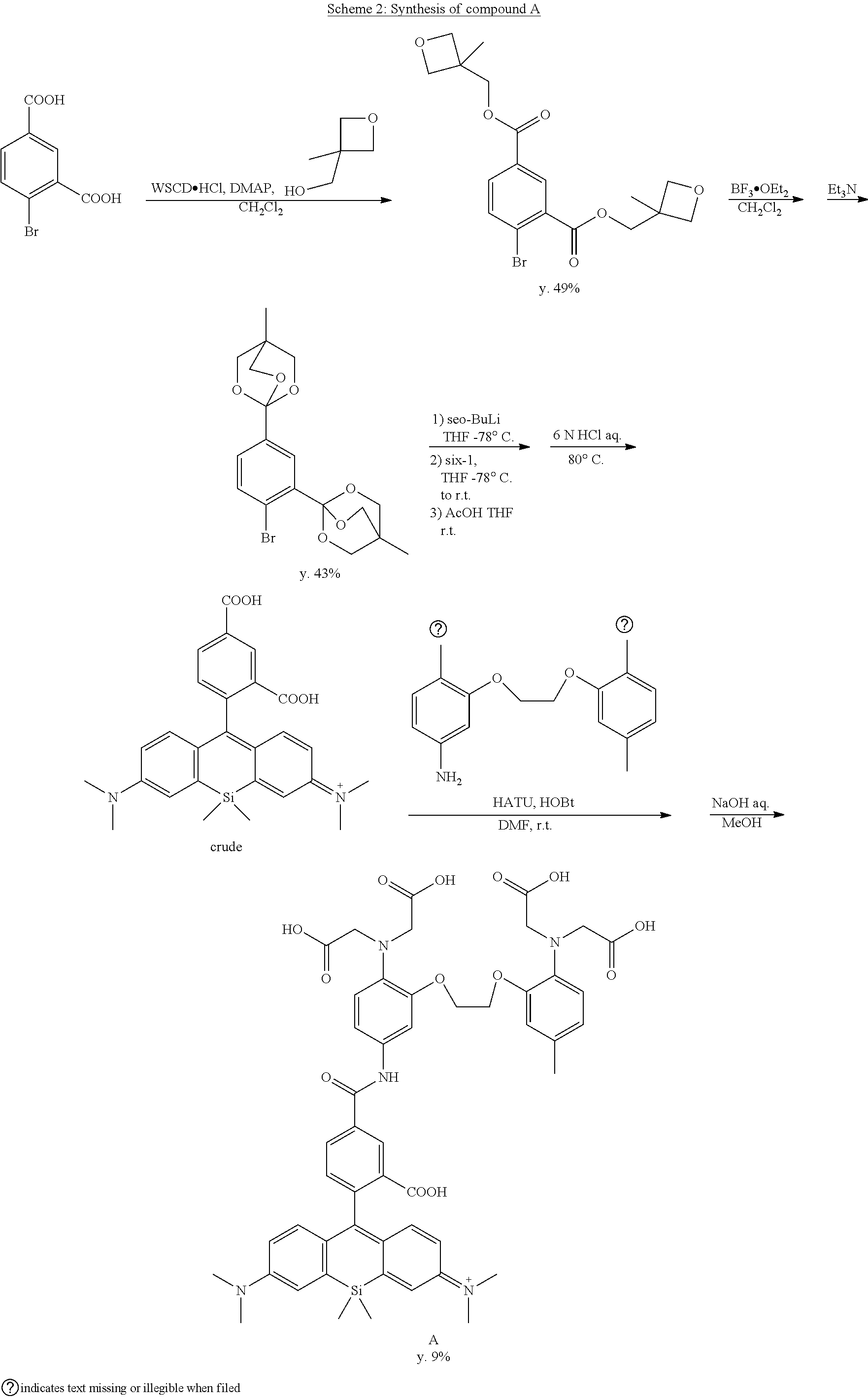

(1) Synthesis of 4-bromo-1,3-benzenedicarboxylic acid 1,3-bis[(3-methyl-3-oxetanyl)methyl]ester

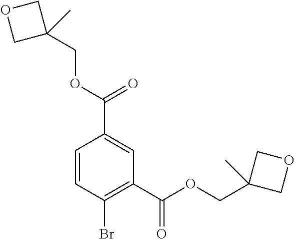 [SMILES: CC1(COC(=O)c2ccc(Br)c(C(=O)OCC3(C)COC3)c2)COC1]

4-Bromoterephthalic acid (5.04 g, 20.53 mmol), WSCD.HCl (8.46 g, 44.32 mmol), DMAP (718 mg, 5.88 mmol), and 3-methyl-3-oxetanemethanol (5.02 mL, 51.18 mmol) were dissolved in dehydrated CH₂Cl₂ and stirred overnight at room temperature. The product was washed with aqueous saturated NaHCO₃ and saturated saline, dried with anhydrous Na₂SO₄, and the solvent was removed under reduced pressure. The residue was purified by silica gel column chromatography (EtOAc/n-hexane=¹/¹), and 2-bromo-1,4-benzenedicarboxylic acid 1,4-bis[(3-methyl-3-oxetanyl)methyl]ester was obtained (4.0:3 g, yield 47%).

¹H NMR (400 MHz, CDCl₃): δ=1.42 (s, 3H), 1.44 (s, 3H), 4.44 (s, 2H), 4.47 (s, 4H), 4.48 (s, 2H), 4.61 (d, J=2.1 Hz, 2H), 4.63 (d, J=1.2 Hz, 2H) 7.78 (d, J=8.4 Hz, 1H), 8.05 (dd, J=8.4 Hz, 1.5 Hz, 2H), 8.31 (d, J=1.5 Hz, 1H). ¹³C NMR (75 MHz, CDCl₃): δ=21.0, 21.2, 39.1, 39.2, 69.9, 70.1, 79.3, 79.4, 121.3, 128.1, 131.1, 1:33.5, 135.1, 136.2, 164.3, 165.6

(2) Synthesis of 1,1′-(2-bromo-1,4-phenylene)bis(4-methyl-2,6,7-trioxabicyclo[2.2.2]octane)

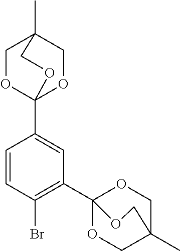 [SMILES: CC12COC(c3ccc(Br)c(C45OCC(C)(CO4)CO5)c3)(OC1)OC2]

Synthesized according to Reference 3 (Grimm J. B., Klein T., Kopek B. G., Shtengel G., Hess H. F., Sauer J., Lavis L. D., Angew. Chem. Int. Ed., 2016, 55, 1723).
(3) Synthesis of 2,4-diCOOHSiR

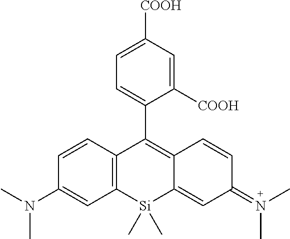

1,1′-(2-Bromo-1,4-phenylene)bis(4-methyl-2,6,7-trioxabicyclo[2.2.2]oxetane) (476 mg, 1.15 mmol) and dehydrated THF (10 mL) were added to a heated and dried flask; after replacing the atmosphere with argon, then cooling to 78° C., 1 M of sec-BuLi (1.15 mmol) was added and stirred for one hour. A solution of SiX-1 (49.0 mg, 0.142 mmol) dissolved in dehydrated THE (10 mL) was added slowly and stirred for 3.5 hours at room temperature. After adding acetic acid (5 mL), the solvent was removed under reduced pressure. The residue was dissolved in 6N hydrochloric acid, then heated and refluxed overnight. After cooling to room temperature and removing the solvent under reduced pressure, the residue was purified by HPLC, and 2,4-diCOOHSiR was obtained (25.5 mg, yield 32%).

¹H NMR (400 MHz, CD₃OD): δ=0.15 (s, 3H), 0.22 (s, 3H), 2.85 (s, 12H), 6.31 (dd, J=7.5 Hz, 2.1 Hz, 2H), 6.49 (d, J=9.8 Hz, 2H), 6.74 (d, J=2.4 Hz, 1H), 6.86 (d, J=2.1 Hz, 1H), 6.98 (d, J=7.8 Hz, 1H.), 7.93 (dd, J=8.0 Hz, 1.7 Hz, 3H), 8.36-8.38 (m, 1H). HRMS (EST+): Calcd for [M]⁺ 483.1896, Found, 473.1877 (−2.0 mmu).

### (4) Synthesis of Compound A

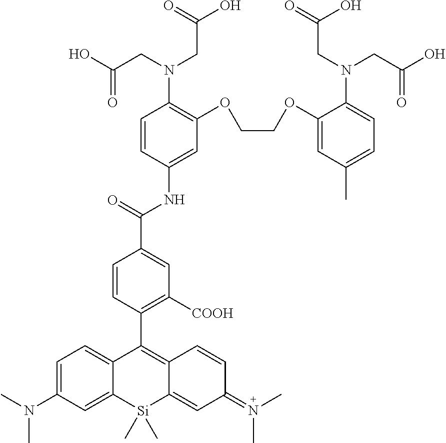

2,4-DiCOOHSiR (25.5 mg, 0.044 mmol), 5-amino-5′-methyl BAPTA tetramethyl ester (20.0 mg, 0.035 mmol), HATU (86.0 mg, 0.226 mmol), and HOBt.H₂O (48.0 mg, 0.30 mmol) were dissolved in DMF (3.0 mL) and stirred overnight. After removing the solvent under reduced pressure, 2N hydrochloric acid was added to the residue which was then extracted with CH₂Cl₂, and the organic layer was washed with saturated saline, dried with anhydrous Na₂SO₄, and the solvent was removed under reduced pressure. The residue was dissolved in 2N NaOH aqueous solution/MeOH (1.5 mL/1.5 mL), stirred for five hours at room temperature, and purified by HPLC, and compound A was obtained (3.1 mg, 3.2 μmol, yield 9%).

¹H NMR (400 MHz, CD₃OD): δ=0.57 (s, 3H), 0.65 (s, 3H), 2.27 (s, 3H), 3.30 (s, 12H), 3.82 (s, 4H), 3.90 (s, 4H), 4.38-4.41 (m, 4H), 6.66 (dd, J=9.0 Hz, 2.7 Hz, 3H), 6.74 (d, J=9.3 Hz, 2H), 6.88 (dd, J=8.3 Hz, 4.9 Hz, 2H), 6.98 (d, J=9.3 Hz, 1H), 7.05 (d, J=2.9 Hz, 2H), 7.37-7.42 (m, 2H), 8.31 (dd, J=8.3 Hz, 1.5 Hz, 1H) 8.51 (d, J=1.0 Hz, 1H).

HRMS (ESI+): Calcd for [M]⁺ 960.3487, Found, 960.3461 (−2.7 mmu). HPLC analysis: eluent: A/B=80/20 to 0/100, 20 min, liner gradient; solvent A: H₂O, 0.1% TFA; solvent B: acetonitrile/H₂O=80/20, 0.1% TFA; flow rate, 1.0 mL/min; detection wavelength 650 nm.

### Synthesis Example 3 (Reference Example)

Compound B was synthesized according to scheme 3 below.

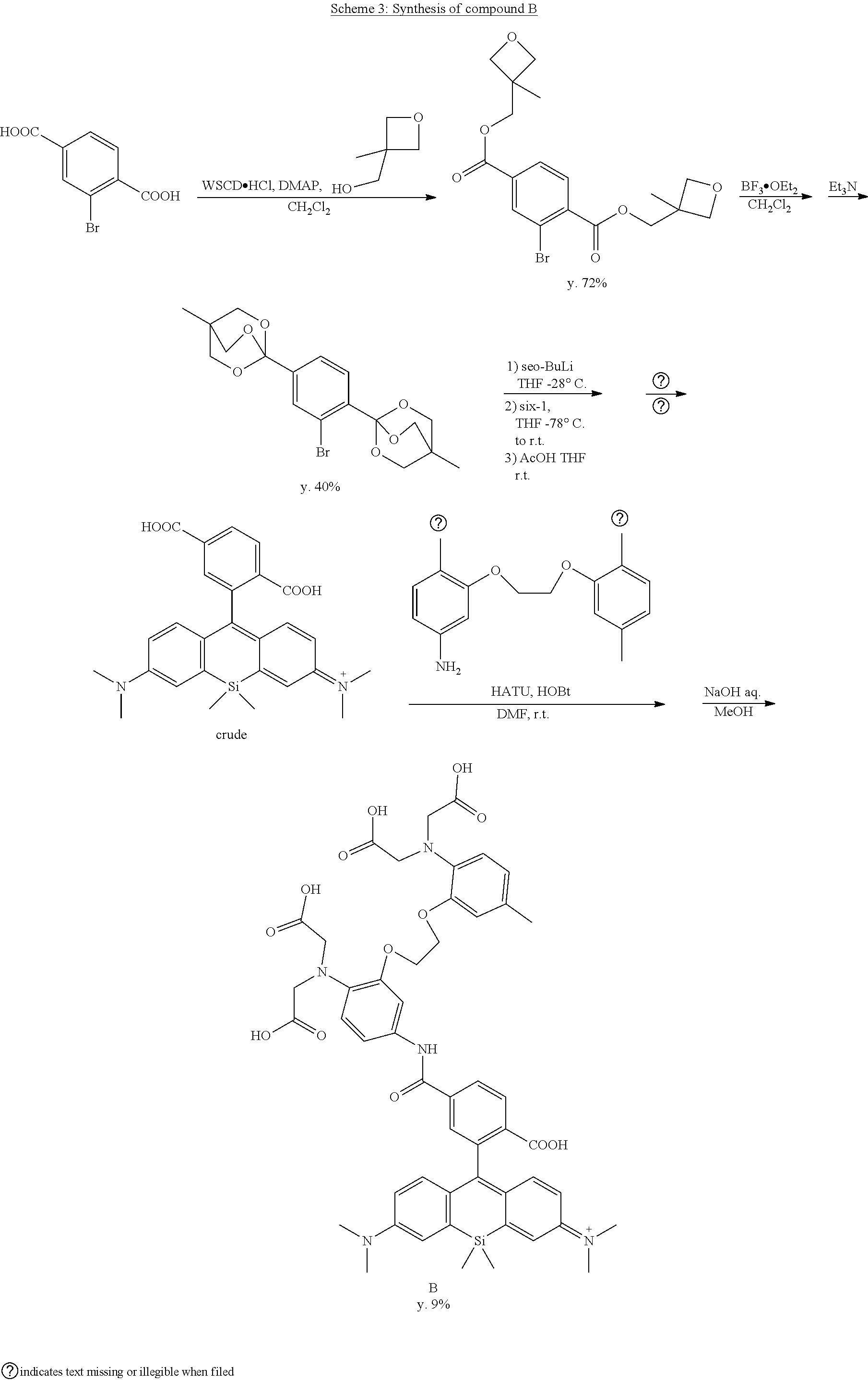

### (10 Synthesis of Compound B

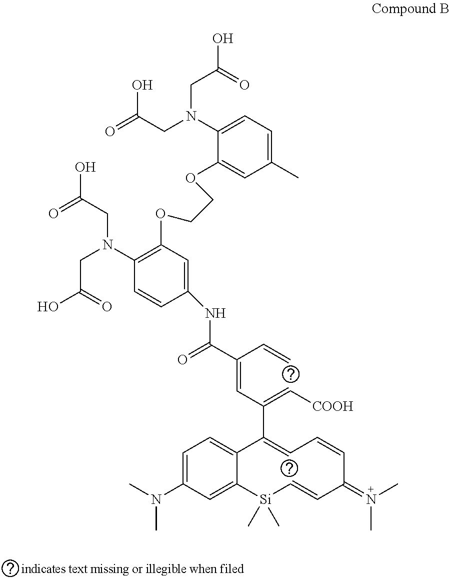

Synthesis from 8.3 mg of 2,5-diCOOHSiR and 23.3 mg of 5-amino-5′-methyl BAPTA tetramethyl ester was performed in the same way as for compound A, and compound B was obtained (1.2 mg, yield 9%).

¹H NMR (400 MHz, CD₃OD): δ=0.53 (s, 3H), 0.64 (s, 3H), 2.22 (s, 3H), 2.94 (s, 12H), 3.79 (s, 4H), 3.92 (s, 4H), 4.29-4.30 (m, 4H), 6.61-6.64 (m, 3H), 6.72 (d, J=8.7 Hz, 2H), 6.79-6.83 (m, 2H), 6.86 (d, J=8.7 Hz, 1H), 7.02 (d, J=2.7 Hz, 2H), 7.27 (d, J=2.3 Hz, 1H), 7.33 (dd, J=8.7 Hz, 2.3 Hz, 1H), 7.79 (s, 1H), 8.03 (d, J=8.2 Hz, 1H) 8.16 (dd, J=8.2 Hz, 1.4 Hz, 1H). HRMS (ESI+): Calcd for [MP]⁺ 960.3487, Found, 960.3507 (2.0 mmu). HPLC analysis: eluent: A/B=80/20 to 0/100, 20 min, liner gradient; solvent A: H₂O, 0.1% TFA; solvent B: acetonitrile/H₂O=80/20, 0.1% TFA; flow rate, 1.0 mL/min; detection wavelength 650 nm.

### Synthesis Example 4

Compound C of the present invention was synthesized according to scheme 4 below.

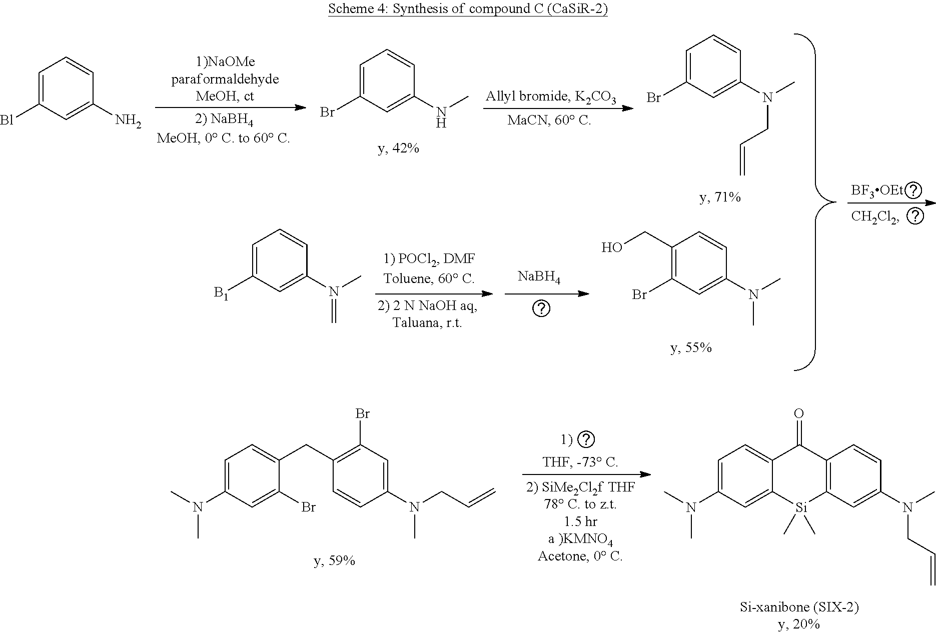

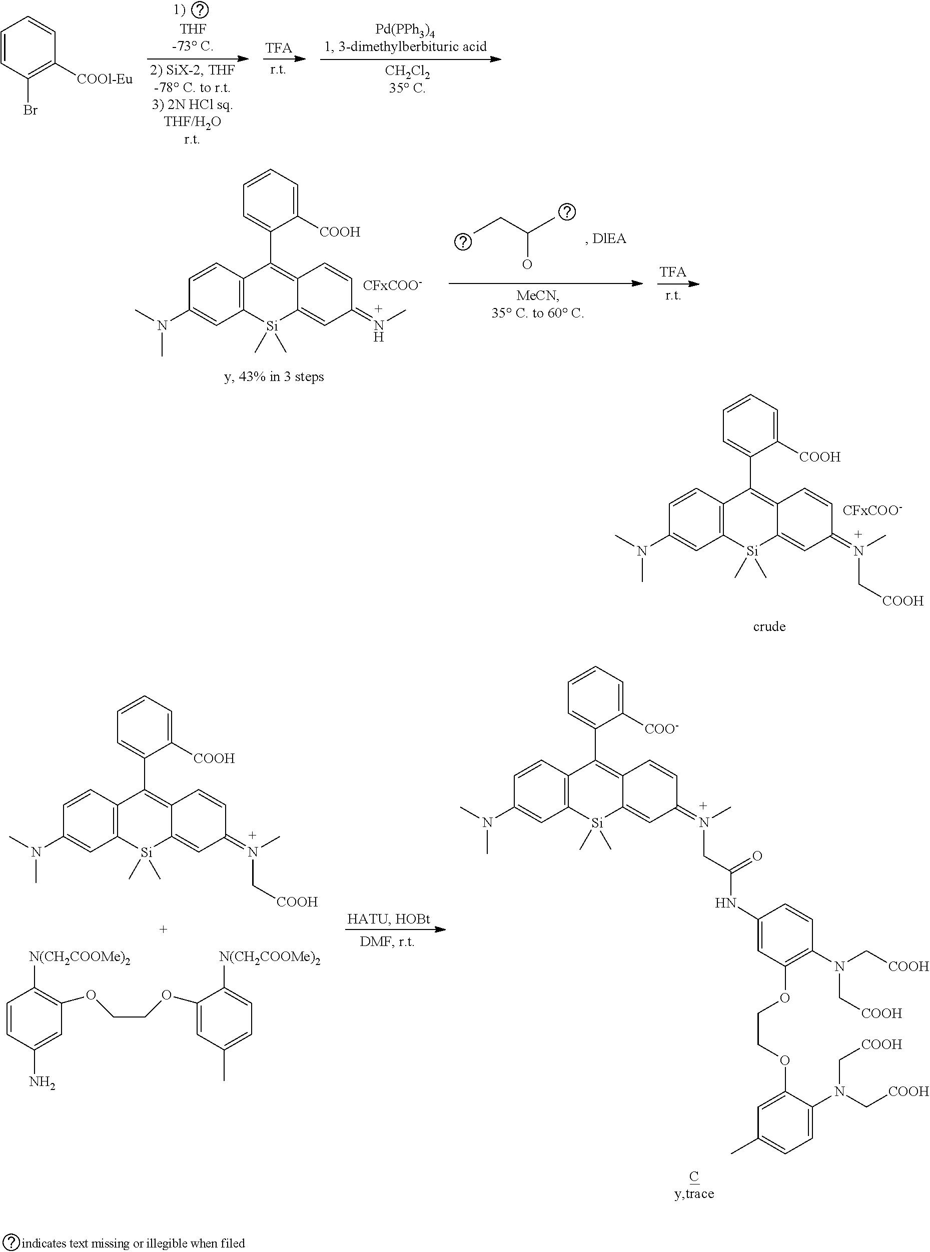

(1) Synthesis of N-methyl-3-bromoaniline

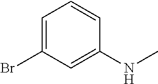

3-Bromoaniline (5.00 g, 29.1 mmol) was dissolved in MeOH (90 mL), and NaOMe (11.25 g, 208 mmol) and paraformaldehyde (13.1 g, 437 mmol) were added and stirred overnight at room temperature. NaBH₄ that had been cooled to 0° C. was added slowly, followed by stirring for two hours at 80° C. After adding 2N NaOH aqueous solution, extraction was performed by CH₂C₂; the organic layer was washed with saturated saline and dried with anhydrous Na₂SO₄, and the solvent was removed under reduced pressure. The residue was purified by silica gel column chromatography (EtOAc/n-hexane=¼), and N-methyl-3-bromoaniline was obtained (2.27 g, 12.4 mol, yield 42).

¹H NMR (300 MHz, CDCl₃): δ=2.81 (s, 3H), 3.78 (br, 1H), 6.51 (dd, J=8.1, 2.2 Hz, 1H), 6.73-6.74 (m, 1H), 6.80 (d, J=8.1 Hz, 1H), 6.99-7.04 (m, 1H). ¹³NMR (75 MHz, CDCl₃): δ=30.5, 111.2, 114.7, 119.9, 123.3, 130.4, 150.5.

(2) Synthesis of N-allyl-N-methyl-3-bromoaniline

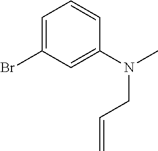

N-methyl-3-bromoaniline (2.27 g, 12.2 mmol), K₂CO₃ (4.20 g, 30.4 mol), and allyl bromide (4.00 g, 33.1 mmol) were dissolved in MeCN (50 mL) and stirred overnight at 80° C. After cooling to room temperature and filtering, the solvent of the filtrate was removed under reduced pressure. The remaining oil droplets were purified by silica gel column chromatography (CH₂Cl₂/n-hexane=½), and N-allyl-N-methyl-3-bromoaniline was obtained (2.02 g, 8.94 mol, yield 73%).

¹H NMR (300 MHz, CDCl₃): δ=2.93 (s, 3H), 3.89-3.91 (m, 2H), 5.10-5.18 (m, 2H), 5.74-5.87 (m, 1H), 6.60 (dd, J=8.4 Hz, 2.6 Hz, 1H), 6.78-6.81 (m 2H), 7.05 (m, 1H). ¹³C NMR (100 MHz, CDCl₃): δ=38.1, 55.1, 110.9, 115.0, 116.5, 119.1, 123.5, 130.4, 133.1, 150.7.

(3) Synthesis of N,N-dimethyl-3-bromo-4-hydroxymethylaniline

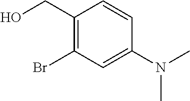

DMF (2 mL, 25.8 mol) and POCl₃ (2.6 mL, 28.0 mmol) were stirred at 100° C., N,N-dimethyl-3-bromoaniline (5.02 g, 25.1 mmol) dissolved in toluene (130 ml) was added thereto and stirred overnight at 100° C. The solution was cooled to room temperature, and 2N NaOH aqueous solution was added and stirred for two hours. The product was extracted with CH₂Cl₂, the organic layer was washed with saturated saline and dried by Na₂SO₄, and the solvent, was removed under reduced pressure. The residue was dissolved in MeOH (100 mL), and NaBH₄ was added slowly at 0° C. and stirred for 5.5 hours. H₂O was added to stop the reaction; the product was extracted with CH₂Cl₂, the organic layer was washed with saturated saline and dried with anhydrous Na₂SO₄, and the solvent was removed under reduced pressure. The residue was purified by silica gel column chromatography (AcOEt/n-hexane=¼), and N,N-dimethyl-3-bromo-4-hydroxymethylaniline was obtained (3.20 g, 13.9 mol, yield 55%).

¹H NMR (300 MHz, CDCl₃): δ=1.87 (t, J=6.6 Hz, 1H), 2.94 (s, 6H), 4.64 (d, J=5.9 Hz, 2H), 6.63 (dd, J=8.4, 2.6 Hz), 6.88 (d, J=2.9 Hz, 1H), 7.25 (d, J=8.8 Hz, 1H). ¹³C NMR. (75 MHz, CDCl₃): δ=40.3, 65.1, 111.4, 116.0, 124.4, 127.0 130.4, 151.1.

(4) Synthesis of (2-bromo-4-N,N-dimethyl) (2-bromo-4-N′-allyl-N′-methyl)methane

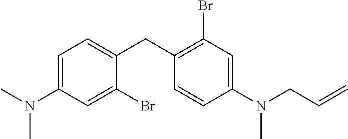 [SMILES: C=CCN(C)c1ccc(Cc2ccc(N(C)C)cc2Br)c(Br)c1]

N-allyl-N-methyl-3-bromoaniline (958 mg, 4.24 mmol) and N,N-dimethyl-3-bromo-4-hydroxymethylaniline (650 mg, 2.83 mmol), and BF₃.OEt₂ (452 mmol, 423 mol) were dissolved in CH₂Cl₂ (10 mL) and stirred overnight at room temperature. H₂O was added to stop the reaction; the product was extracted with CH₂Cl₂, the organic layer was washed with saturated saline and dried with anhydrous Na₂SO₄, and the solvent was removed under reduced pressure. The residue was purified by silica gel column chromatography (CH₂Cl₂/n-hexane=⅓), and (2-bromo-4-N,N-dimethyl) (2-bromo-4-N′-allyl-N′-methyl)methane was obtained (876 mg, 2.00 mmol, yield 69%).

¹H NMR (300 MHz, CDCl₃): δ=2.88 (s, 9H), 3.83-3.85 (m, 2H), 3.98 (s, 2H), 5.10-5.15 (m, 2H), 5.74-5.83 (m, 1H), 6.52-6.58 (m, 2H), 6.80-6.85 (m, 2H), 6.90-6.93 (m, 2H). ¹³C. NMR (100 MHz, CDCl₃): δ=38.1, 39.9, 40.6, 55.2, 111.8, 111.9, 116.1, 116.3, 116.5, 125.7, 127.0, 127.2, 130.9, 133.4, 149.0, 150.1.

(5) Synthesis of N-allyl-N,N′,N′-trimethyl-Si-xanthone (SiX-2)

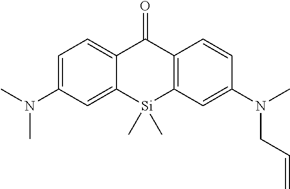 [SMILES: C=CCN(C)c1ccc2c(c1)[Si](C)(C)c1cc(N(C)C)ccc1C2=O]

N-allyl-N,N′,N′-trimethyl-Si-xanthone was obtained (187 mg, yield 20%) from (2-bromo-4-N,N-dimethyl) (2-bromo-4-N′-allyl-N′-methyl)methane (1.17 g) according to Reference 2 in the same way as N,N,N′,N′-tetramethyldiamino-Si-xanthone.

¹H NMR (300 MHz, CDCl₃): δ=0.45 (s, 6H), 3.08 (s, 3H), 3.09 (s, 6H), 4.03-4.05 (m, 2H), 5.15-5.21 (m, 2H), 5.80-5.92 (m, 1H, 6.78-6.85 (m, 4H), 8.37 (d, J=4.5 Hz, 1H), 8.40 (d, J=4.5 Hz, 1H)

¹³C NMR (75 MHz, CDCl₃): δ=−1.1, 38.0, 40.0, 54.6, 113.1, 113.2, 114.2, 114.4, 116.5, 129.6, 131.5, 131.6, 132.7, 110.4, 140.5, 150.6, 151.4, 185.2.

### (6) Synthesis of 2-COOHSiR630

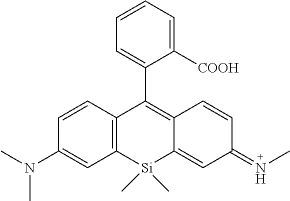

Tert-butyl-2-bromobenzoate (395 mg, 1.54 mmol) and dehydrated THF (3 mL) were added to a heated and dried flask; after replacing the atmosphere with argon and cooling to −78° C., 1 M of sec-BuLi (1.3 mmol) was added and stirred for four minutes. SiX-2 (94.0 mg, 0.267 mmol) dissolved in dehydrated THF (4 mL) was added slowly; after stirring for two hours at room temperature, 2N HCl aq. (5 mL) was added and stirred for 30 minutes. The product was extracted with CH₂Cl₂, the organic layer was washed with saturated saline and dried with anhydrous Na₂SO₄, and the solvent was removed under reduced pressure. The residue was dissolved in TFA (5 mL) and stirred for three hours. The TFA was removed, the residue was lightly purified by silica gel column chromatography, a fraction containing an intermediate (ESI-MS (+): 455) was recovered, and the solvent was removed under reduced pressure. The residue was dissolved in CH₂Cl₇ (30 mL), and 1,3-dimethylbarbituric acid (144 mg, 0.923 mmol) and Pd(PPh₃)₄ (99 mg, 0.086 mmol) were added and stirred overnight at 35° C. After removing the solvent under reduced pressure, the product was purified by HPLC, and 2-COOHSiR630 trifluoroacetate was obtained (60.6 mg, 0.115 mmol, yield. 43%).

¹H NMR (300 MHz, CD₃OD): δ=0.56 (s, 3H), 0.61 (s, 3H), 3.03(s, 3H), 3.28 (s, 6H), 6.61 (d, J=9.5 Hz, 2H), 6.74 (d, J=9.5 Hz, 1H), 6.97-7.00 (m, 2H), 7.20-7.32 (m, 3H), 7.67 (m, 2H), 8.23 (d, J=7.3 Hz, 1H). HRMS (ESI+): Calcd for [M]⁺ 415.1842, Found, 415.1843 (+0.1 mmu).

### (7) Synthesis of 2-COOHSiR650-COOH

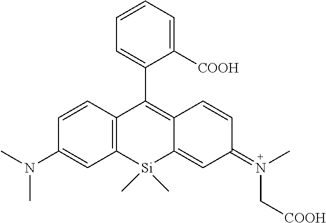

2-COOHSiR630 trifluoroacetate (22.0 mg, 41.6 μmol) was dissolved in tert-butyl bromoacetate (13.8 μL, 102 μmol), and DIEA (14.2 μL, 82.5 μmol) was added and stirred overnight at 35° C. After removing the solvent under reduced pressure, the residue was dissolved in TFA (5 mL) and stirred for 1.5 hour at room temperature. After removing the TFA, the residue was lightly purified by HPLC, and crude 2-COOH650-COOH (10.3 mg) was obtained. This compound was used without further modification in the next reaction.

### (8) Synthesis of Compound C (CaSiR-2)

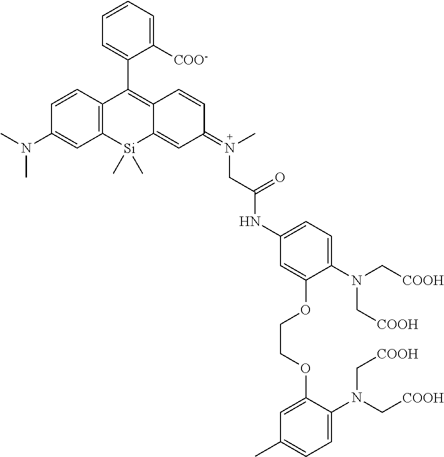

Synthesis from 2-COOH650-COOH (3.7 mg) and 5-amino-5′-methyl BAPTA tetramethyl ester (6.0 mg) was performed in the same way as for compound A, and compound C was obtained (0.1 mg, yield 2%).

¹H NMR (400 MHz, CD₂OD): δ=0.50 (s, 3H), 0.57 (s, 3H), 2.22 (s, 3H), 2.90 (s, 6H), 3.08 (s, 3H), 3.46 (s, 4H), 2.50 (s, 4H), 4.09 (s, 2H), 4.22-4.26 (m, 4H), 6.57 (dd, J=9.1 Hz, 3.2 Hz, 1H), 6.61-6.62 (m, 2H), 6.65 (d, J=8.7 Hz, 1H), 6.68 (d, J=9.1 Hz, 1H), 6.73 (d, J=1.4 Hz, 1H), 6.89 (d, J=8.2 Hz, 1H), 6.94 (d, J=8.7 Hz, 1H), 6.98 (dd, J=1.4 Hz, 1.4 Hz, 2H), 7.02 (dd, J=9.4 Hz, 2.5 Hz, 1H), 7.21 (d, J=2.3 Hz, 1H), 7.25 (q, J=7.8 Hz, 1H), 7.59 (ddd, J=7.5 Hz, 7.5 Hz, 1.2 Hz 1H), 7.71 (ddd, J=7.5 Hz, 7.5 Hz, 1.2 Hz, 1H), 7.89 (d, J=7.8 Hz, 1H), 8.49 (br, 1H).

HRMS (ESI+): Calcd for [M]+ 960.3487, Found, 960.3453 (−3.6 mmu). HPLC analysis: eluent: A/B=80/20 to 0/100, 20 min, liner gradient; solvent A: H₂O, 0.1% TFA; solvent B: acetonitrile/H₂O=80/20, 0.1% TEA; flow rate, 1.0 mL/min; detection wavelength 650 nm.

### Example 1

### Evaluation of Compounds A-C as Ca²⁺ Probes

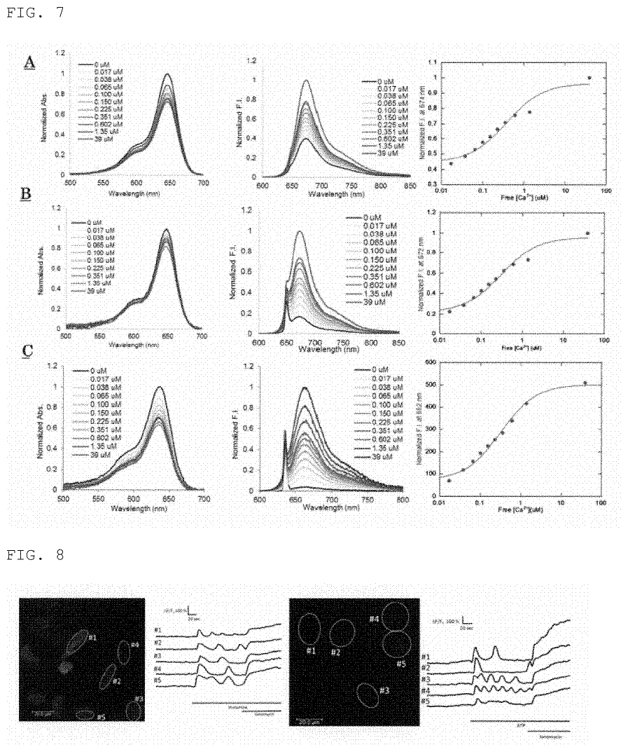

To investigate whether the synthesized compounds A, B, and C can function as Ca²⁺ probes, the absorption and fluorescence spectra were measured in solutions of various Ca²⁺ concentrations. The results are shown in FIG. 7.

FIG. 7 represents the absorption spectra (left), emission spectra (center), and fluorescence spectra (right) of 1 μM of compounds A, B, and C in the presence of various concentrations (0, 0.017, 0.038, 0.065, 0.100, 0.150, 0.225, 0.351, 0.602, 1.35, and 39 mM) of free Ca²⁺ in pH 7.2 30 mM 3-(N-morpholino)propanesulfonic acid (MOPS) containing 100 nM of KCl and 10 nM of ethylene glycol tetraacetic acid (EGTA). The excitation wavelengths were 646 nm (A), 648 nm (B), and 635 nm (C).

The right side of FIG. 7 shows plots of the fluorescence intensity of 1 μM of compounds A, B, and C in the presence of various concentrations of free Ca²⁺ in pH 7.2 30 mM MOPS containing 100 nM KCl and 10 nM of EGTA.

Also, Table 1 shows the photophysical properties of compounds A, B, and C.

TABLE 1

| | In 0 μM free [Ca²⁺] | | | In 39 μM free [Ca²⁺] | | | | |
|---|---|---|---|---|---|---|---|---|
| | buffer | | | buffer | | | | |
| | λ_{abs} | λ_{fl} | | λ_{abs} | λ_{fl} | | Activation | K_{d} |
| | (nm) | (nm) | Φ_{fl} | (nm) | (nm) | Φ_{fl} | Ratio | (μM) |
| A | 646 | 673 | 0.10 | 646 | 674 | 0.25 | ×2.5 | 0.30 |
| B | 648 | 671 | 0.04 | 647 | 672 | 0.29 | ×7.3 | 0.37 |
| C | 637 | 664 | 0.01 | 636 | 661 | 0.26 | ×26 | 0.31 |

The fluorescence intensity of all of the synthesized compounds A, B, and C rose as the Ca²⁺ concentration rose, and these compounds were understood to function as Ca²⁺ probes. However, significant differences were seen in the fluorescence quantum yield and activation ratio in the absence of Ca²⁺. Whereas ϕ_{f1} was 0.10 and a 2.5-fold activation ratio was seen with compound A, ϕ_{f1} was 0.01 and a 26-fold activation ratio was seen with compound C. This result is thought to reflect the distance between the BAPTA structure which is the electron donor and the xanthene ring which is the electron acceptor, as hypothesized, and compound C was thought to be more greatly quenched by PeT because the number of bonds between the two was the smallest and the distance the closest. Compound. C was designated as CaSiR-2 below, and further studies were conducted.

### Example 2

### Live Cell Application of a Novel Ca²⁺ Probe

Next, CaSiR-2 was applied to HeLa cells to confirm that CaSiR-2 remains in the cytoplasm without accumulating in specific organelles in an intracellular environment. A probe CaSiR-2AM in which the carboxylic acid of the BAPTA structure was protected by an AM group was synthesized to impart cell membrane permeability to CaSiR-2.

### Synthesis Example 5

CaSiR-2AM (Compound D) was synthesized according to scheme 5 below.

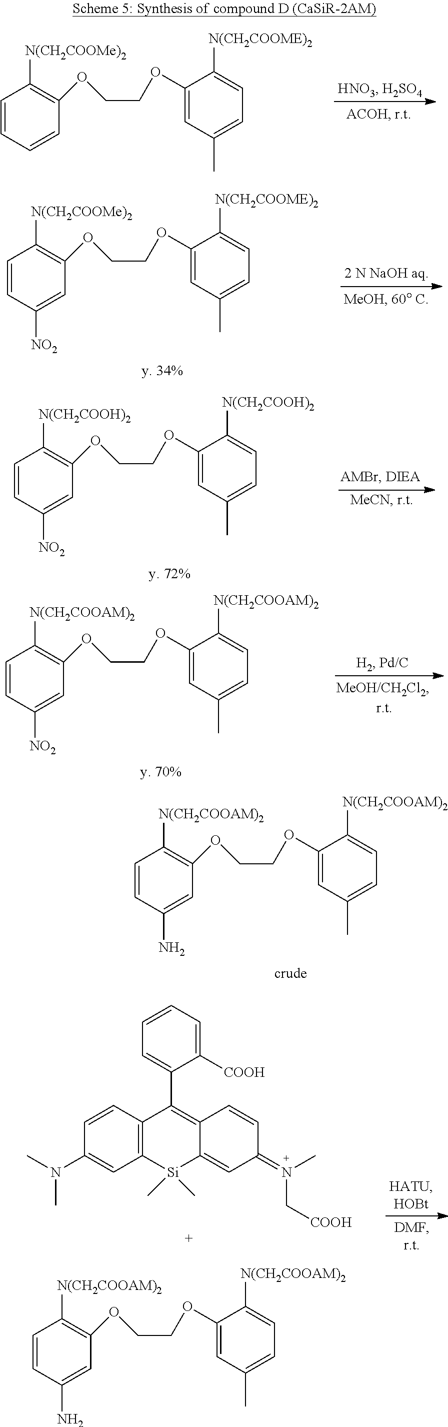

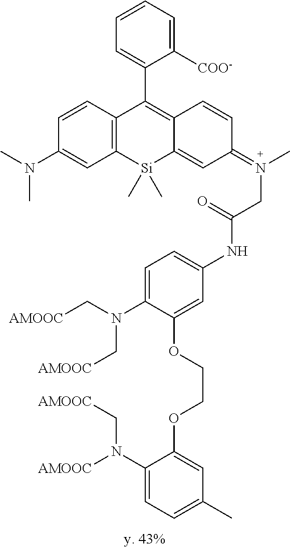

(1) Synthesis of 5-methyl-5′-nitro BAPTA

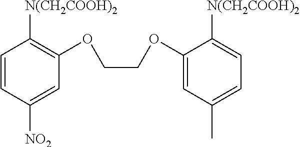

5-Nitro BAPTA tetramethyl ester (259 mg, 0.438 mol) was dissolved in MeOH (5 mL), and 2N NaOH aq. (8 mL) was added and stirred overnight at room temperature. The solution was neutralized by 2N HCl, and the solvent was removed under reduced pressure. The residue was purified by HPLC, and 5-methyl-5′-nitro BAPTA was obtained (170 mg, 0.318 mmol, yield 72%).

¹H NMR (400 MHz, CD₃OD): δ=2.27 (s, 3H), 4.12 (s, 4H), 4.24-4.34 (m, 8H), 6.67-6.71 (m, 3H), 6.77 (d, J=7.3 Hz, 1H), 7.73 (d, J=2.9 Hz, 1H), 7.83 (dd, J=8.8, 2.9 Hz, 1H). ¹³C NMR (75 MHz, CD₃OD): δ=21.0, 68.1, 69.3, 109.5, 115.7, 116.5, 119.2, 120.8, 122.8, 134.5, 137.1, 141.7, 147.0, 149.8, 151.9, 174.4, 175.3. HRMS (ESI⁺): Calcd for [M+Na]⁺, 558.1336, Found, 558.1340 (+0.4 mmu).

(2) Synthesis of 5-methyl-5′-nitro BAPTA tetraacetoxymethyl ester

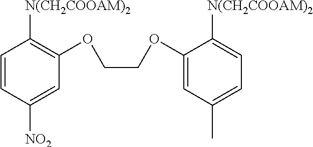

5-methyl-5′-nitro BAPTA (147.8 mg, 0.276 mmcl) was dissolved in MeCN (5 mL), and FIFA (420 μL, 2.41 mmol) and bromomethyl acetate (120 μL, 1.2 mmol) were added and stirred overnight. After acidifying by adding acetic acid, the product was purified by HPLC, and 5-methyl-5′-nitro BAPTA tetraacetoxymethyl ester was obtained (158 mg, 0.192 mmol, yield 70%).

¹H NMR (300 MHz, CD₂Cl₂): δ=2.03 (s, 6H), 2.05 (s, 6H), 2.27 (s, 3H), 4.13 (s, 43), 4.28-4.31 (m, 6H), 4.36-4.39 (m, 2H), 5.58 (s, 4H), 5.61 (s, 4H), 6.70-6.75 (m, 3H), 6.80 (d, J=8.1 Hz), 7.76 (d, J=2.9 Hz, 1H), 7.81 (dd, J=9.2, 2.6 Hz, 1H). ¹³C NMR (75 MHz, CDCl₃): δ=20.5, 20.8, 53.3, 53.4, 66.7, 67.7, 79.0, 79.4, 108.2, 114.9, 116.4, 118.0, 120.4, 122.3, 133.0, 136.2, 141.3, 144.6, 148.7, 150.3, 169.1, 169.3, 169.3, 169.9. HRMS (ESI⁺): Calcd for [M+Na]⁺, 846.2181, Found, 846.2173 (−0.8 mmu).

(3) Synthesis of 5-methyl-5′-amino BAPTA tetraacetoxymethyl ester

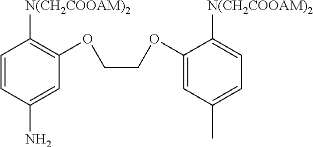

5-methyl-5′-nitro BAPTA tetraacetoxymethyl ester (147.9 mg, 0.179 mmol) was dissolved in EtOH (5 mL) and CH₂Cl₂ (5 mL), and Pd/C (10%) was added and stirred for three hours at room temperature in the presence of hydrogen. The Pd/C was removed by filtration, and the solvent was removed under reduced pressure. The residue was lightly purified by HPLC, and crude 5-methyl-5′-amino BAPTA tetraacetoxymethyl ester was obtained (77.0 mg).

### (4) Synthesis of CaSiR-2AM

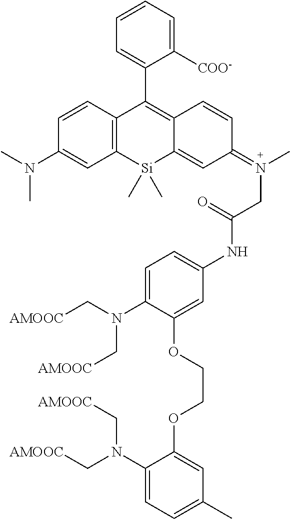

2-COOH650-COOH (4.1 mg, 7.0 μmol), 5-amino-5′-methyl BAPTA tetraacetoxymethyl ester (35.0 mg, 41 μmol), HATU (15.7 mg, 41.3 μmol), and HOBt.H₂O (3.6 mg, 23.5 μmol) were dissolved in DMF (2.0 ml) and stirred overnight at room temperature. After neutralizing by adding acetic acid, the product was purified by HPLC, and CaSiR-2AM was obtained (4.0 mg, 3.06 μmol, yield 43%).

¹H NMR (400 MHz, CD₃OD): δ=0.55 (s, 3H), 0.62 (s, 3H), 2.00 (s, 6H), 2.03 (s, 6H), 2.28 (s, 3H), 2.98 (s, 6H), 3.17 (s, 3H), 4.13-4.17 (m, 10H), 4.26 (s, 4H), 5.58 (s, 4H), 5.60 (s, 4H), 6.63-6.84 (m, 8H), 7.01-7.08 (m, 3H), 7.29-7.33 (m, 2H), 7.64-7.68 (m, 1H), 7.75-7.78 (m, 1H), 7.96 (d, J=7.8 Hz, 1H). HRMS (ESI+): Calcd for [M]⁺ 1248.4332, Found, 1248.4289 (−4.3 mmu). HPLC analysis: eluent: A/B=80/20 to 0/100, 20 min, liner gradient; solvent A: H₂O, 0.1 TFA; solvent B: acetonitrile/H₂O=80/20, 0.1% TFA; flow rate, 1.0 mL/min; detection wavelength 650 nm.

Imaging studies in HeLa cells were carried out using the synthesized CaSiR-2AM.

For the studies, HeLa cells were incubated for 30 minutes together with 3 μM of CaSiR-2AM in HBSS containing 0.3% DMSO. The dye was then washed off three times, and imaging was begun.

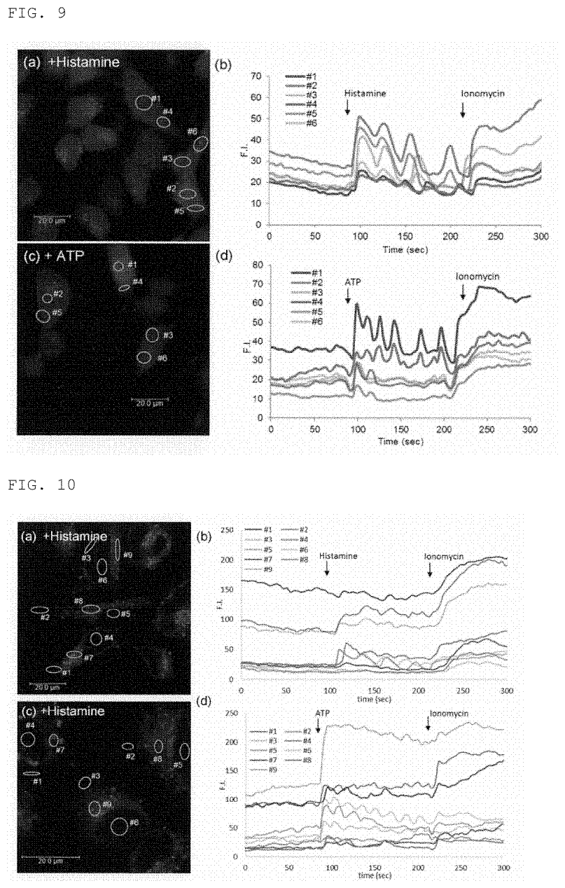

As a result, although a small amount of fluorescence can be seen localized in points from inside the cells, the majority of the probe was understood to be distributed in the cytoplasm, as expected (FIGS. 8 and 9). Also, the localization that can be seen inside the cells is thought to be localization to lysosomes based on the punctiform localization.

Next, whether the Ca²⁺ concentration fluctuations generated inside the cell due to stimulation from outside the cell can be captured by CaSiR-2AM was studied. (FIG. 8). Specifically, after loading HeLa cells with CaSiR-2AM, histamine or ATP was added to the extracellular fluid to generate calcium oscillations inside the cells, and whether these oscillations could be captured as changes in fluorescence intensity was studied. Also, ionomycin which is a calcium ionophore was finally added and whether elevation of the intracellular Ca²⁺ concentration was seen was investigated.

Histamine activates phospholipase C via H1 receptors on the cell membrane of HeLa cells, and phospholipase C hydrolyzes PIP₂ to produce IP₃ (Reference 13: Hill S. J., Ganellin C. R., Timmerman H., Schwartz J. C., Shankley N. P., Young J. M., Schunack W., Levi R., Haas H. L., Pharmacol Rev., 1997, 49, 253-278). IP₃ binds to IP₃ receptors on the endoplasmic reticulum, and Ca²⁺ channels on the endoplasmic reticulum open and release Ca²⁺ (Reference 14: R. Y. Tsien., Annu. Rev. Biophys. Bioeng., 1983, 12, 91-116). Calcium oscillations due to histamine stimulation occur mainly through a pathway via H1 receptors (Reference 15: Zhu D. M., Tekle E., Huang C. Y., Chock P. B., J. Biol. Chem., 2000, 275, 6063-6066). On the other hand, ATP is known to activate phospholipase C via P2Y₁ receptors on the cell membrane in stem cells from human bone marrow (Reference 16: Kawano S., Otsu K., Kuruma A., Shoji S., Yanagida E., Muto Y., Yoshikawa F., Hirayama Y. Mikoshiba K., Furuichi T., Cell Calcium, 2006, 39, 313-324) and radial glia (Reference 17: Barrack D. S., Thul R., Owen M. R., J. Theor. Biol, 2014, 347, 17-32) and to raise the intracellular Ca²⁺ concentration by the same pathway as histamine thereafter.

When histamine (1 μM) or ATP (100 μM) was added 90 seconds after the start of fluorescence observation, changes in the state of the intracellular fluorescence intensity were observed. Also, when ionomycin (5 μM) was added 210 seconds after the start of fluorescence observation, a rise in intracellular fluorescence was observed. Based on the above results, observation of the intracellular Ca²⁺ concentration fluctuations due to extracellular stimulation by CaSiR-2AM was successful. Specifically, CaSiR-2AM was shown to be a near-infrared fluorescent Ca²⁺ sensor capable of capturing calcium oscillations in the cytoplasm.

Furthermore, FIG. 8 shows changes in fluorescence of the ROI of individual cells 1 to 5, and the images were taken at excitation and emission wavelengths of 650 nm/1670-750 nm.

Next, CaSiR-2AM was compared with the existing near-infrared fluorescent probe CaSiR-1AM. CaSiR-1AM is a probe the fluorescence of which is kept very low (ϕ<0.001) and that exhibits very large activation in the absence of Ca²⁺, while on the other hand localization to the lysosomes has been suggested (Reference 5: Egawa T., Hanaoka K., Koide Y., Ujita S., Takahashi H., Ikegaya Y., Matsuki N., Terai T., Ueno T., Komatsu T., Nagano T., J. Am. Chem. Soc., 2011, 133, 13157-14159).

FIGS. 9 and 10 show the results of tracing the fluorescence signal of CaSiR-2AM and CaSiR-1AM by circling the ROI of each organelle. In this study, the vertical axis uses the fluorescence intensity rather than the fluorescence change rate in the analysis results to understand the fluorescence intensity before adding stimulation.

The left side of FIG. 9 shows a visualization of histamine (a) or ATP (c) an beta cells using CaSiR-2 AM, and the right side shows the induced calcium oscillations. HeLa cells were cultured together with 3 μM of CaSiR-2AM and 0.03% Pluronic in HBSS containing 0.45% DMSO for 30 minutes at 37° C. The dye was then washed off three times, and imaging was begun.

The addition conditions of histamine or ATP and ionomycin were the same as in FIG. 8. The change in fluorescence of the ROI of individual cells (#1-#3: nucleus; #4-#6: cytoplasm; #7: background) 1 to 7 is shown. Also, the images were taken at excitation and emission wavelengths of 650 nm/670-750 nm.

The left side of FIG. 10 shows a visualization of histamine (a) or ATP (c) in beta cells using CaSiR-1AM, and the right side shows the induced calcium oscillations, HeLa cells were cultured together with 3 μM of CaSiR-1AM and 0.03% Pluronic in HBSS containing 0.45% DMSO for 30 minutes at 37° C. The dye was then washed off three times, and imaging was begun.

Histamine or ATP and ionomycin were added under the same conditions as in FIG. 8. The change in fluorescence of the ROI of individual cells (#1-43: nucleus; #4-#6: cytoplasm; #7-#9: lysosome; #10: background) 1 to 10 is shown. Also, the images were taken at excitation and emission wavelengths of 650 nm/670-750 nm.

With both CaSiR-2AM and CaSiR-1AM, calcium oscillations of fluorescence intensity reaching about twice that before stimulation at maximum were observed in the nucleus (#1-#3) and cytoplasm (#4-#6), but with CaSiR-1AM it was understood that the calcium oscillations in the lysosome (#7-#9) where the most dye accumulated and the fluorescence intensity was high were not observed to the extent of in the nucleus and cytoplasm. In other words, CaSiR-1 was understood to be incapable of high-sensitivity measurement when capturing intracellular calcium oscillations due to high background fluorescence derived from the probe accumulated in the lysosomes.

The reason why CaSiR-1AM emits fluorescence in the lysosomes has been explained by previous research (Reference 18: Lloyd-Evans E., Morgan A. J., He X., Smith D. A., Elliot-Smith E., Sillence D. J., Churchill G. C., Schuchman E. H., Galione A., Platt F. M., Nat. Med., 2008, 14, 1247-1255). Specifically, the reason is because Ca²⁺ is present from the start in the lysosomes in a concentration of several hundred μM at which the fluorescence of CaSiR-1AM disappears. Therefore, CaSiR-1AM can be a useful probe when observing the calcium concentration of lysosomes, but CaSiR-2AM that has the property of accumulating in the cytoplasm is clearly more suitable for capturing at high sensitivity the fluctuations in the intracellular calcium ion concentration which are important in calcium signaling.

### Example 3

### Application of CaSiR-2AM to Ca²⁺ Imaging in Rat Brain Slices

In the field of neuroscience, it is necessary to track the activity of multiple neurons simultaneously to explain brain function, and Ca²⁺ imaging is very important as a basic technique for doing so (Reference 19: Grienberger C., Konnerth A., Neuron, 2012, 73, 862-885; Reference 20: Takahashi N., Sasaki T., Usami A., Matsuki N., Ikegava Y., Neurosci. Res., 2007, 58, 219-225; Reference 21: 21. Losonczy A., Makara J. K., Magree J. C., Nature, 2008, 452, 436-441).

In the past, action potentials associated with neural activity were measured using electrodes. However, the number of neurons that can be measured by such electrophysiological methods is limited and functional multineuron calcium imaging (fMCI), which makes it possible to observe multiple neurons at a single cell level at good spatial and temporal resolution, has come to be used as a method for observing actual neural activity since actual neural activity is established on a huge neural network composed of multiple neurons (Reference 22: Mizunuma M., Ikegaya Y., Folia Pharmacol. Jpn., 2009, 134, 17-21).

The neurons that constitute the brain carry out spontaneous neural activity which is called spontaneous firing. And, in association with the firing, voltage-dependent Ca²⁺ channels in the brain neurons open and Ca²⁺ flows into the cell. In other words, by introducing a Ca²⁺ probe into neurons and observing the fluorescence using fMCI, the activity of multiple neurons within the field of view can be observed simultaneously by substituting the changes in the fluorescence of the probe, and which neuron carries out activity at what timing can be observed visually. Therefore, whether the spontaneous firing phenomenon can be observed in neurons by the compound of the present invention CaSiR-2AM was investigated.

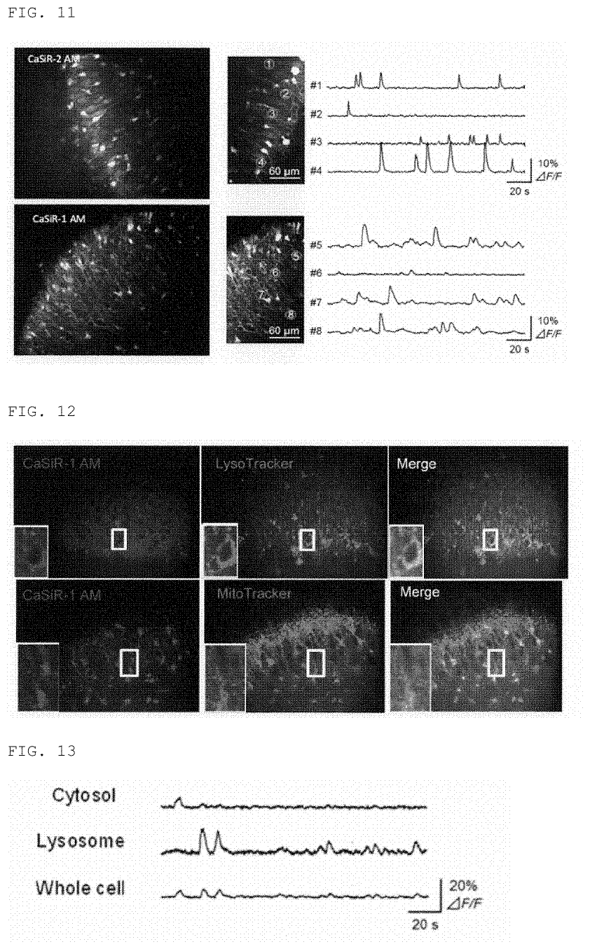

The probe was loaded by the simple method of immersing a rat brain slice in artificial cerebrospinal fluid to which CaSiR-2AM had been added, fluorescence imaging was carried out, and whether CaSiR-2AM is taken up into the neurons and whether spontaneous firing of the neurons can be observed at a single cell level was confirmed by comparison with CaSiR-1AM (FIG. 11).

A comparison of the left-hand drawings in FIG. 11 reveals intracellular localization to differ between CaSiR-2AM and CaSiR-1AM. CaSiR-2AM distributes the dye to the entire cell and a state in which the cytoplasm is stained can be observed, but a state of localization to some of the intracellular organelles is observed with CaSiR-1AM. Also, as relates to calcium concentration fluctuations in neurons, while CaSiR-2AM captured the calcium response of individual neurons at a high S/N ratio, CaSiR-1AM was understood to be unable to carry out. imaging at a high S/N ratio because the signal rise was sluggish and the baseline was unstable.

It is very interesting that the state of the calcium response changes markedly depending on differences in the localization of the probe, and the reason that the signal did not sharpen with CaSiR-1AM was thought to be the simultaneous capture of calcium fluctuations in organelles where the dye was localized. The results of this study illustrated again that it is important for the Ca²⁺ probe to accumulate in the cytoplasm in order to capture spontaneous firing in neurons.

Next, to confirm in which organelles CaSiR-¹ is actually localized, co-staining was carried out by adding LysoTracker Green (75 nM), which is a lysosome stain reagent, or MitoTracker (200 nM), which is a mitochondrial stain reagent, simultaneously with probe loading of rat brain slices (FIG. 12).

Based on the fluorescence imaging results, LysoTracker was observed to merge better than MitoTracker for the localization of CaSiR-1AM in neurons. Therefore, CaSiR-1AM was understood to localize to the lysosomes in studies in rat brain slices in the same way as in cultured cells.

Also, when the site-by-site traces of CaSiR-1AM were compared, it was understood that the whole-cell trace is obtained by addition of the traces of the structures thought to be lysosomes and the cytoplasm. (FIG. 13). Io other words, when observing neural firing using mouse brain slices, CaSiR-1AM captures mainly the calcium fluctuations in the lysosomes in addition to calcium concentration fluctuations in the cytoplasm of the neurons, thereby raising the baseline and making it impossible to capture the neural firing at high sensitivity. The study clarified that the reason why CaSiR-2AM can capture neural firing at high sensitivity in mouse brain slice systems is that CaSiR-2AM captures only the calcium concentration fluctuations in the cytoplasm associated with neural firing by accumulating in the cytoplasm.

1. A compound represented by the following general formula (I) or a salt thereof: 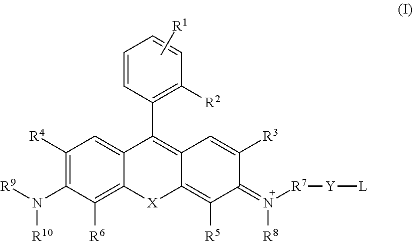
where:
R¹ is a hydrogen atom or one to four of the same or different monovalent substituents present on the benzene ring, and R¹ may be the same or different;
R² is an anionic functional group, a C1-10 alkyl group, or a C1-10 alkoxy group;
R³ and R⁴ are, each independently, a hydrogen atom, a C1-6 alkyl group, or a halogen atom;
R⁵ and R⁶ are, each independently, a hydrogen atom, a C1-6 alkyl group, or a halogen atom;
X is SiR¹¹R¹², GeR¹¹R¹², SnR¹¹R¹², CR¹¹R¹², SO₂, or POR¹³,
R¹¹ and R¹² are, each independently, a C1-6 alkyl group or an aryl group,
R¹³ is a C1-6 alkyl group or an optionally substituted phenyl group;
R⁷ is a C1-6 alkylene group;
R⁸ is a hydrogen atom or a C1-6 alkyl group,
R⁸ optionally forms, together with R⁵, a five- to seven-membered heterocyclyl or heteroaryl containing a nitrogen atom to which R⁸ is bonded, and optionally contains from one to three heteroatoms selected from the group consisting of an oxygen atom, nitrogen atom, and sulfur atom as ring members, and the heterocyclyl or heteroaryl may be substituted by a C1-6 alkyl, C2-6 alkenyl, or C2-6 alkynyl, C6-10 aralkyl group, or C6-10 alkyl-substituted alkenyl group;
R⁹ and R¹⁰ are, each independently, a hydrogen atom or a C1-6 alkyl group,
R⁹ and R¹⁰ together may form a four- to seven-membered heterocyclyl containing a nitrogen atom to which R⁹ and R¹⁰ are bonded,
R⁹ or R¹⁰, or both R⁹ and R¹⁰, together with R⁴, R⁶, respectively, may form a five- to seven-membered heterocyclyl or heteroaryl containing a nitrogen atoms to which R⁹, R¹⁰ are bonded, optionally containing one to three heteroatoms selected from the group consisting of an oxygen atom, nitrogen atom, and sulfur atom as ring members, and the heterocyclyl or heteroaryl may be substituted by a C1-6 alkyl, C2-6 alkenyl, or C2-6 alkynyl, C6-10 aralkyl group, or C6-10 alkyl-substituted alkenyl group;
Y, when present, is a spacer:
L is a substituent which acts as a capturing group for a substance to be measured.

2. The compound or salt thereof according to claim 1, wherein the anionic functional group of R² is selected from a hydroxyl group, carboxy group, sulfo group, C1-10 hydroxyalkyl group, C1-10 alkyl group having a carboxy group, or C1-10 alkoxy group having a carboxy group.

3. The compound or salt thereof according to claim 1, wherein the capturing group is a capturing group for capturing a proton, a metal ion, a low-oxygen environment, an active oxygen species, nitrogen monoxide, hydrogen peroxide, singlet oxygen, or a pH environment.

4. The compound or salt thereof according to claim 3, wherein the metal ion is selected from a zinc ion, magnesium ion, sodium ion, potassium ion, or calcium ion.

5. The compound or salt thereof according to claim 1, wherein the capturing group is a capturing group for capturing a calcium ion.

6. The compound or salt thereof according to claim 1, wherein Y is an amide, ester, or thiourea.

7. The compound or salt thereof according to claim 1, wherein L is a capturing group for capturing a calcium ion represented by general formula (1) below. 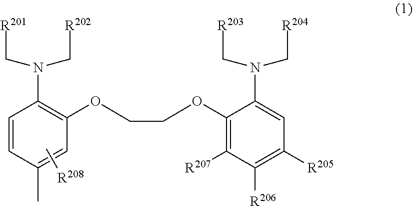
wherein, R²⁰¹, R²⁰², R²⁰³, and R²⁰⁴ are, each independently, a carboxy group, an alkyl group having a carboxy group, an ester group, an optionally substituted alkyl ester group, or a salt thereof;
R²⁰⁵, R²⁰⁶, and R²⁰⁷ are, each independently, a hydrogen atom, a halogen atom, a C1-6 alkyl group, a methoxy group, or a nitro group;
R²⁰⁸ is a hydrogen atom or one to three of the same or different monovalent substituents present on the benzene ring.

8. The compound or salt thereof according to claim 1, wherein L is a capturing group for capturing a calcium ion represented by formula (2) below: 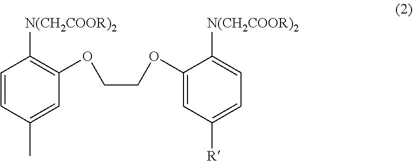
wherein, R is hydrogen or —CH₂OCOCH₃, each R may be the same or different:
R′ is a methyl group, a methoxy group, or a fluorine atom.

9. The compound or salt thereof according to claim 1, wherein R² is a carboxy group.

10. The compound or salt thereof according to claim 1, wherein R⁷ is selected from a methylene group or an ethylene group and R⁸ is selected from a methyl group or an ethyl group.

11. The compound or salt thereof according to claim 1, wherein R⁹ and R¹⁰ are, each independently, selected from a methyl group or an ethyl group.

12. The compound or salt thereof according to claim 1, wherein R⁷ is a methylene group, and R⁸, R⁹, and R¹⁰ are all methyl groups.

13. The compound or salt thereof according to claim 1, wherein R¹ are all hydrogen atoms.

14. A compound represented by formula (3) below, or a salt thereof. 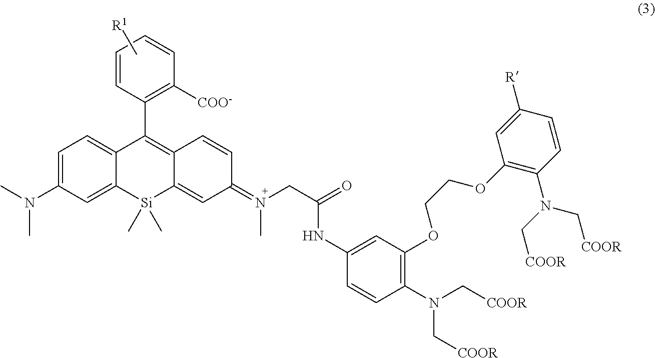
wherein, R is hydrogen or —CH₂OCOCH₃, each R may be the same or different:
R′ is a methyl group, a methoxy group, or a fluorine atom, R¹ is as defined in general formula (I).

15. A fluorescent probe containing a compound or salt thereof according to claim 1.

16. A method for measuring a substance to be measured, wherein the method comprises:
(a) bringing the compound or salt thereof according to claim 1 into contact with a substance to be measured and
(b) measuring the fluorescence intensity of the compound after capture of the substance to be measured generated in said (a) above.

17. The method according to claim 16, wherein the substance to be measured is a calcium ion.
